(19) United States
(12) Patent Application Publication
(10) Pub. No.: US 2025/0136533 A1
(43) Pub. Date: May 1, 2025

(54) LITHIUM CARBIDE MATERIAL

(71) Applicant: Group14 Technologies, Inc., Woodinville, WA (US)

(72) Inventors: Henry R. Costantino, Woodinville, WA (US); Abirami Dhanabalan, Bothell, WA (US); Avery J. Sakshaug, Snohomish, WA (US); Brandon Rotondo, Woodinville, WA (US); Mitchell Keller, Woodinville, WA (US); Nigel Leytham, Woodinville, WA (US)

(21) Appl. No.: 18/930,820

(22) Filed: Oct. 29, 2024

Related U.S. Application Data
(60) Provisional application No. 63/594,319, filed on Oct. 30, 2023.

(51) Int. Cl. C07C 5/35; C01B 32/935; G01N 15/06

(52) U.S. Cl. CPC C07C 5/35; C01B 32/935; G01N 15/06; C01P 2002/50; C01P 2004/61; C01P 2004/62; C01P 2006/12; C07C 2527/22

(57) ABSTRACT

Disclosed herein are applications for using lithium carbide materials including reducing agents, radiocarbon dating, and acetylene storage materials.

### BACKGROUND

### Technical Field

Embodiments of the present disclosure generally relate to particles comprising compounds of Group 14 elements, including but not limited to carbon, and Group 1 elements, including but not limited to lithium and methods of manufacturing the same and applications related thereto. In particular, embodiments include use as a reducing agent, acetylene storage and production, and radiocarbon dating application.

### Description of the Related Art

Lithium acetylide compounds are generally formed by reacting acetylene (C₂H₂) with lithium metal. The most prominent use of lithium carbide is as an intermediate in radiocarbon dating. For instance, typically a carbon-containing sample is burned to produce CO₂ and then the carbon-containing sample is reacted with lithium metal to produce a Li₂C₂ byproduct. From the Li₂C₂ species a mass spectrometry friendly species can be produced (e.g., acetylene or benzene) by reacting the sample with lithium. Calcium carbide is a common acetylide known in the art for use in the production of acetylene whereby the reaction of calcium carbide with water produces acetylene and calcium hydroxide.

CaC₂(s)+2H₂0(aq)→C₂H₂(g)+Ca(OH)₂(aq)

Lithium carbide, e.g., Li₂C₂, materials (e.g., FIG. 1) are most commonly radiocarbon dating procedures. For instance, in the radiocarbon dating example, lithium carbide is produced as an intermediate in the radiocarbon dating process. The present material may be employed in such a process. For instance, lithium carbide may be used to create mass spectrometry friendly species such as acetylene and benzene that can then be correlated to an age of the material being studied.

An additional application for the lithium carbide material is in the storage of acetylene. In this application, acetylene can be produced from lithium carbide materials providing a more efficient storage medium.

Another application for the lithium carbide compounds is as a reducing agent. The lithium carbide materials provide an advantage over alternative organolithiums available on the market. For instance, lithium hydride, a common reducing agent, is often contaminated with elemental lithium making stoichiometric additions difficult to achieve in practice. Further, the solid nature of the lithium carbide compounds allows for easier handling than the pyrophoric liquid alternatives such as butyllithiums. There is a need for a reducing agent that optimizes selectivity in the reduction reaction or to balance reactivity with reagent stability. For instance, available heteroatom doped lithium carbide compounds would be less desirable as it may have impacts on final product purity as the residual metal compounds may be harder to separate.

### BRIEF SUMMARY

Disclosed herein are materials and methods related to lithium carbide materials. Some embodiments comprising the use of lithium carbide material include (1) as a reducing agent, (2) acetylene storage, and (3) material for use in radiocarbon dating applications. The lithium carbide materials in these instances may contain stoichiometric excess lithium or carbon that deviates from a 1:1 molar stoichiometry.

Lithium carbide is not commonly used as a reducing agent. Typically, reducing agents are substances that donate electrons to other compounds, causing them to gain electrons and undergo reduction. Reducing agent solutions derived from alkali metals mixed in a solution of polycyclic aromatic hydrocarbons (arenes) and ether solvent have been traditionally prepared from the alkali element in a metallic (neutral charge) state. While effective, this entails an additional upstream synthesis step to form the metal using high temperature molten salt electrolysis which is energy intensive, and require significant infrastructure to keep the alkali metal product stable (under inert atmosphere) and costly to ship because of the highly reactive nature of the metal. Embodiments disclosed herein solves these challenges by forming the reactive alkali cation and arene anion in-situ in an electrolytic cell containing a soluble salt of the alkali element (e.g., LiCl, KCl, etc.) as the alkali feedstock rather than the reactive metal. This circumvents the high temps needed for molten salt electrolysis and high costs associated with shipping highly reactive alkali metals. However, the lithium carbide materials as disclosed herein have utility as reducing agents.

In addition, the lithium carbide materials disclosed here may have utility in the storage and production of acetylene for applications such as radiocarbon dating. For instance, Li₂C₂ may react with water to produce a lithium hydroxide and acetylene (C₂H₂). This reaction may be particularly useful as an intermediate reaction in radiocarbon dating.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIG. 1. shows an atomic lattice diagram schematic of a lithium carbide compound.

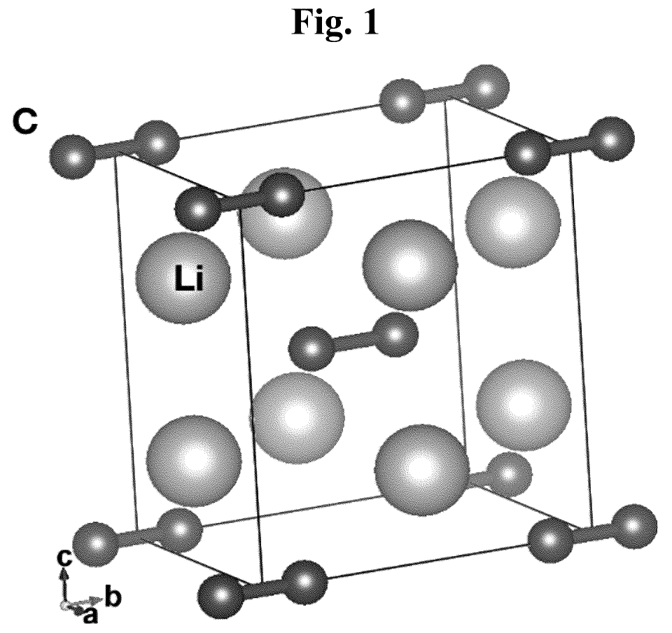

FIG. 2. shows a differential scanning calorimetry heat flow curve of the reaction between a lithium hydride and a carbon powder.

FIG. 3A. shows a differential scanning calorimetry heat flow curve of a lithium hydride.

FIG. 3B. shows a differential scanning calorimetry heat flow curve of a carbon power.

FIG. 4. shows a differential scanning calorimetry heat flow curve of an exemplary lithium carbide compound (LCC8) exhibiting a melting point of 448° C.

FIG. 5. shows an X-ray diffraction pattern of an exemplary lithium carbide material (LCC7) indicating a Li₂C₂ (lmmm) phase is present.

FIG. 6. shows an X-ray diffraction pattern of an exemplary lithium carbide material (LCC8) indicating the presence of both Li₂C₂ (lmmm) and Li₂C₂ (Fm-3m) phases.

FIG. 7. shows Voltage profiles indicating an extraction capacity of lithium carbide compounds up to 4.6V (vs. Li/Li⁺).

FIG. 8. shows a Voltage profile of lithium carbide (LCC16) indicating an extraction capacity up to 4.2V and insertion capacity down to 0.5V (vs. Li/Li+) at different capacity limitations.

FIG. 9. shows a voltage profile of LCC8 showing the effect of carbon nanotube (CNT) addition to the electrode formulation on electrochemical performance of lithium carbide.

### DETAILED DESCRIPTION

In the following description, certain specific details are set forth in order to provide a thorough understanding of various embodiments. However, one skilled in the art will understand that the disclosure may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### A. Porous Scaffold Materials

For the purposes of embodiments of the current disclosure, lithium may be reacted with a porous scaffold. In this context, the porous scaffold can comprise various materials. In some embodiments the porous scaffold material primarily comprises carbon, for example hard carbon. Other allotropes of carbon are also envisioned in other embodiments, for example, graphite, amorphous carbon, diamond, C60, carbon nanotubes (e.g., single and/or multi-walled), graphene and/or carbon fibers. The introduction of porosity into the carbon material can be achieved by a variety of means. For instance, the porosity in the carbon material can be achieved by modulation of polymer precursors, and/or processing conditions to create said porous carbon material, and described in detail in the subsequent section.

In other embodiments, the porous scaffold comprising carbon can be derived from a polymer precursor material. To this end, a wide variety of polymers are envisioned in various embodiments to have utility, including, but not limited to, inorganic polymers, organic polymers, and additional polymers. Examples of organic polymers includes, but are not limited to, sulfur-containing polymers such polysulfides and polysulfones, low density polyethylene (LDPE), high density polyethylene (HDPE), polypropylene (PP), polyvinyl chloride (PVC), polystyrene (PS), nylon, nylon 6, nylon 6,6, teflon (Polytetrafluoroethylene), thermoplastic polyurethanes (TPU), polyureas, poly(lactide), poly(glycolide) and combinations thereof, phenolic resins, polyamides, polyaramids, polyethylene terephthalate, polychloroprene, polyacrylonitrile, polyaniline, polyimide, poly(3,4-ethylenedioxythiophene) polystyrene sulfonate (PEDOT:PSS), polymerized polydivinylbenzene, and others known in the arts. The organic polymer can be synthetic or natural in origin. In some embodiments, the polymer is a polysaccharide, such as sucrose, starch, cellulose, cellobiose, amylose, amylopectin, gum Arabic, lignin, and the like. In some embodiments, the polysaccharide is derived from the caramelization of mono- or oligomeric sugars, such as fructose, glucose, sucrose, maltose, raffinose, and the like.

Concomitant with the myriad variety of polymers envisioned with the potential to provide a porous carbon scaffold, various processing approaches are envisioned in various embodiments to achieve said porosity. In this context, general methods for imparting porosity into various materials are myriad, as known in the art, including, but certainly not limited to, methods involving emulsification, micelle creation, gasification, dissolution followed by solvent removal (for example, lyophilization), axial compaction and sintering, gravity sintering, powder rolling and sintering, isostatic compaction and sintering, metal spraying, metal coating and sintering, metal injection molding and sintering, and the like. Other approaches to create a porous polymeric material, including creation of a porous gel, such as a freeze dried gel, aerogel, and the like are also envisioned.

### B. Porous Carbon Scaffold Materials

Methods for preparing porous carbon materials from polymer precursors are known in the art. For example, methods for preparation of carbon materials are described in U.S. Pat. Nos. 7,723,262, 8,293,818, 8,404,384, 8,654,507, 8,916,296, 9,269,502, 10,590,277, and 10,711,140, the full disclosures of which are hereby incorporated by reference in their entireties for all purposes.

Accordingly, in one embodiment the present disclosure provides a method for preparing any of the carbon materials or polymer gels described above. The carbon materials may be synthesized through pyrolysis of either a single precursor, for example a saccharide material such as sucrose, fructose, glucose, dextrin, maltodextrin, starch, amylopectin, amylose, lignin, gum Arabic, and other saccharides known in the art, and combinations thereof. Alternatively, the carbon materials may be synthesized through pyrolysis of a complex resin, for instance formed using a sol-gel method using polymer precursors such as phenol, resorcinol, bisphenol A, urea, melamine, and other suitable compounds known in the art, and combinations thereof, in a suitable solvent such as water, ethanol, methanol, and other solvents known in the art, and combinations thereof, with cross-linking agents such as formaldehyde, hexamethylenetetramine, furfural, and other cross-linking agents known in the art, and combinations thereof. The resin may be acid or basic, and may contain a catalyst. The catalyst may be volatile or non-volatile. The pyrolysis temperature and dwell time can vary as known in the art.

In some embodiments, the methods comprise preparation of a polymer gel by a sol gel process, condensation process or crosslinking process involving monomer precursor(s) and a crosslinking agent, two existing polymers and a crosslinking agent or a single polymer and a crosslinking agent, followed by pyrolysis of the polymer gel. The polymer gel may be dried (e.g., freeze dried) prior to pyrolysis; however drying is not necessarily required.

The target carbon properties can be derived from a variety of polymer chemistries provided the polymerization reaction produces a resin/polymer with the necessary carbon backbone. Different polymer families include novolacs, resoles, acrylates, styrenes, urethanes, rubbers (neoprenes, styrene-butadienes, etc.), nylons, etc. The preparation of any of these polymer resins can occur via a number of different processes including sol gel, emulsion/suspension, solid state, solution state, melt state, etc. for either polymerization or crosslinking processes.

In some embodiments the reactant comprises phosphorus. In certain other embodiments, the phosphorus is in the form of phosphoric acid. In certain other embodiments, the phosphorus can be in the form of a salt, wherein the anion of the salt comprises one or more phosphate, phosphite, phosphide, hydrogen phosphate, dihydrogen phosphate, hexafluorophosphate, hypophosphite, polyphosphate, or pyrophosphate ions, or combinations thereof. In certain other embodiments, the phosphorus can be in the form of a salt, wherein the cation of the salt comprises one or more phosphonium ions. The non-phosphate containing anion or cation pair for any of the above embodiments can be chosen for those known and described in the art. In the context, exemplary cations to pair with phosphate-containing anions include, but are not limited to, ammonium, tetraethylammonium, and tetramethylammonium ions. In the context, exemplary anions to pair with phosphate-containing cations include, but are not limited to, carbonate, dicarbonate, and acetate ions.

In some embodiments, the reactant comprises sulfur. In certain other embodiments, the sulfur is in the form of sulfuric acid. In certain other embodiments, the sulfur can be in the form of a salt, wherein the anion of the salt comprises one or more sulfate, sulfite, bisulfide, bisulfite, hypothiocyanite, sulfonium, S-methylmethionine, thiocarbonate, thiocyanate, thiophosphate, thiosilicate, or trimethylsulfonium, or combinations thereof.

In some embodiments, the catalyst comprises a basic volatile catalyst. For example, in one embodiment, the basic volatile catalyst comprises ammonium carbonate, ammonium bicarbonate, ammonium acetate, ammonium hydroxide, or combinations thereof. In a further embodiment, the basic volatile catalyst is ammonium carbonate. In another further embodiment, the basic volatile catalyst is ammonium acetate.

In still other embodiments, the method comprises admixing an acid. In certain embodiments, the acid is a solid at room temperature and pressure. In some embodiments, the acid is a liquid at room temperature and pressure. In some embodiments, the acid is a liquid at room temperature and pressure that does not provide dissolution of one or more of the other polymer precursors.

In one embodiment a spherical polydivinylbenzene spheres are produced by precipitation polymerization, pyrolyzed, and activated by the methods described herein.

In one embodiment, a porous carbon can be prepared by pyrolysis of a fluorine containing polymer (e.g., polyvinylidenine fluoride) by heating the material to 600 C under an inerting gas such as nitrogen flowing in a horizontal tube furnace. The material was allowed to cool for 30 minutes and subsequently cooled to room temperature prior to removal from the furnace. The resulting carbonized material was attrition milled to less than 25-micron particle size distribution and used to prepare electrodes. The porous carbon prepared by this method is rich in fluorine which facilitates formation of lithium fluoride in the initial stage of electrochemical plating of the lithium metal in a lithium ion battery, thereby increasing the lithiophilicity and reduces detrimental dendrite growth in the battery.

In certain embodiments, the polymer precursor components are blended together and subsequently held for a time and at a temperature sufficient to achieve polymerization. One or more of the polymer precursor components can have particle size less than about 20 mm in size, for example less than 10 mm, for example less than 7 mm, for example, less than 5 mm, for example less than 2 mm, for example less than 1 mm, for example less than 100 microns, for example less than 10 microns. In some embodiments, the particle size of one or more of the polymer precursor components is reduced during the blending process.

The blending of one or more polymer precursor components in the absence of solvent can be accomplished by methods described in the art, for example ball milling, jet milling, Fritsch milling, planetary mixing, and other mixing methodologies for mixing or blending solid particles while controlling the process conditions (e.g., temperature). The mixing or blending process can be accomplished before, during, and/or after (or combinations thereof) incubation at the reaction temperature.

Reaction parameters include aging the blended mixture at a temperature and for a time sufficient for the one or more polymer precursors to react with each other and form a polymer. In this respect, suitable aging temperature ranges from about room temperature to temperatures at or near the melting point of one or more of the polymer precursors. In some embodiments, suitable aging temperature ranges from about room temperature to temperatures at or near the glass transition temperature of one or more of the polymer precursors. For example, in some embodiments the solvent free mixture is aged at temperatures from about 20° C. to about 600° C., for example about 20° C. to about 500° C., for example about 20° C. to about 400° C., for example about 20° C. to about 300° C., for example about 20° C. to about 200° C. In certain embodiments, the solvent free mixture is aged at temperatures from about 50 to about 250° C.

The reaction duration is generally sufficient to allow the polymer precursors to react and form a polymer, for example the mixture may be aged anywhere from 1 hour to 48 hours, or more or less depending on the desired result. Typical embodiments include aging for a period of time ranging from about 2 hours to about 48 hours, for example in some embodiments aging comprises about 12 hours and in other embodiments aging comprises about 4-8 hours (e.g., about 6 hours).

In certain embodiments, an electrochemical modifier is incorporated during the above described polymerization process. For example, in some embodiments, an electrochemical modifier in the form of metal particles, metal paste, metal salt, metal oxide or molten metal can be dissolved or suspended into the mixture from which the gel resin is produced.

Exemplary electrochemical modifiers for producing lithium carbide materials may fall into one or more than one of the chemical classifications. In some embodiments, the electrochemical modifier is a lithium salt, for example, but not limited to, lithium fluoride, lithium chloride, lithium carbonate, lithium hydroxide, lithium benzoate, lithium bromide, lithium formate, lithium hexafluorophosphate, lithium iodate, lithium iodide, lithium perchlorate, lithium phosphate, lithium sulfate, lithium tetraborate, lithium tetrafluoroborate, and combinations thereof.

In certain embodiments, the electrochemical modifier comprises a metal, and exemplary species includes, but are not limited to aluminum isopropoxide, manganese acetate, nickel acetate, iron acetate, tin chloride, silicon chloride, and combinations thereof. In certain embodiments, the electrochemical modifier is a phosphate compound, including but not limited to phytic acid, phosphoric acid, ammonium dihydrogen phosphate, and combinations thereof. In certain embodiments, the electrochemical modifier comprises silicon, and exemplary species includes, but are not limited to silicon powders, silicon nanotubes, polycrystalline silicon, nanocrystalline silicon, amorphous silicon, porous silicon, nano sized silicon, nano-featured silicon, nano-sized and nano-featured silicon, silicyne, black silicon, and combinations thereof.

Electrochemical modifiers can be combined with a variety of polymer systems through either physical mixing or chemical reactions with latent (or secondary) polymer functionality. Examples of latent polymer functionality include, but are not limited to, epoxide groups, unsaturation (double and triple bonds), acid groups, alcohol groups, amine groups, basic groups. Crosslinking with latent functionality can occur via heteroatoms (e.g. vulcanization with sulfur, acid/base/ring opening reactions with phosphoric acid), reactions with organic acids or bases (described above), coordination to transition metals (including but not limited to Ti, Cr, Mn, Fe, Co, Ni, Cu, Zn, Zr, Nb, Mo, Ag, Au), ring opening or ring closing reactions (rotaxanes, spiro compounds, etc.).

Electrochemical modifiers can also be added to the polymer system through physical blending. Physical blending can include but is not limited to melt blending of polymers and/or co-polymers, the inclusion of discrete particles, chemical vapor deposition of the electrochemical modifier and co-precipitation of the electrochemical modifier and the main polymer material.

In some instances, the electrochemical modifier can be added via a metal salt solid, solution, or suspension. The metal salt solid, solution or suspension may comprise acids and/or alcohols to improve solubility of the metal salt. In yet another variation, a polymer gel (either before or after an optional drying step) is contacted with a paste comprising the electrochemical modifier. In yet another variation, a polymer gel (either before or after an optional drying step) is contacted with a metal or metal oxide sol comprising the desired electrochemical modifier.

In addition to the above exemplified electrochemical modifiers, the carbon materials may comprise one or more additional forms (i.e., allotropes) of carbon. In this regard, it has been found that inclusion of different allotropes of carbon such as graphite, amorphous carbon, conductive carbon, carbon black, diamond, C60, carbon nanotubes (e.g., single and/or multi-walled), graphene, carbon fibers, or combinations thereof into the lithium carbide materials is effective to improve the electrochemical properties of the material. The various allotropes of carbon can be incorporated into the lithium carbide materials during any stage of the preparation process described herein. For example, during the solution phase, during the gelation phase, during the curing phase, during the pyrolysis phase, during the milling phase, or after milling. In some embodiments, the second carbon form is incorporated into the compound material by adding the second carbon form before or during polymerization of a polymer gel as described in more detail herein. The polymerized polymer gel containing the second carbon form is then processed according to the general techniques described herein to obtain a carbon material containing a second allotrope of carbon.

In some embodiments, the polymer precursor is a polyvinylbenzene sphere produced by precipitation polymerization. More particularly, the polymer precursor may be polydivinylbenzene microspheres produced through a distillation-precipitation polymerization reaction as known in the art. In other embodiments, the polymer precursor in the low or essentially solvent free reaction mixture is a urea or an amine containing compound. For example, in some embodiments the polymer precursor is urea, melamine, hexamethylenetetramine (HMT) or combination thereof. Other embodiments include polymer precursors selected from isocyanates or other activated carbonyl compounds such as acid halides and the like.

Some embodiments of the disclosed methods include preparation of low or solvent-free polymer gels (and carbon materials) comprising electrochemical modifiers. Such electrochemical modifiers include, but are not limited to nitrogen, silicon, and sulfur. In other embodiments, the electrochemical modifier comprises fluorine, iron, tin, silicon, nickel, aluminum, zinc, or manganese. The electrochemical modifier can be included in the preparation procedure at any step. For example, in some the electrochemical modifier is mixed with the mixture, the polymer phase or the continuous phase.

The porous carbon material can be achieved via pyrolysis of a polymer produced from precursor materials as described above. In some embodiments, the porous carbon material comprises an amorphous activated carbon that is produced by pyrolysis, physical or chemical activation, or combination thereof in either a single process step or sequential process steps.

The temperature and dwell time of pyrolysis can be varied, for example the dwell time can vary from 1 min to 10 min, from 10 min to 30 min, from 30 min to 1 hour, for 1 hour to 2 hours, from 2 hours to 4 hours, from 4 hours to 24 h. The temperature can be varied, for example, the pyrolysis temperature can vary from 200° C. to 300° C., from 250° C. to 350° C., from 350° C. to 450° C., from 450° C. to 550° C., from 540° C. to 650° C., from 650° C. to 750° C., from 750° C. to 850° C., from 850° C. to 950° C., from 950° C. to 1050° C., from 1050° C. to 1150° C., from 1150° C. to 1250° C. In some embodiments, the pyrolysis temperature varies from 650° C. to 1100° C. The pyrolysis can be accomplished in an inert gas, for example nitrogen, or argon.

In some embodiments, an alternate gas is used to further accomplish carbon activation. In certain embodiments, pyrolysis and activation are combined. Suitable gases for accomplishing carbon activation include, but are not limited to, carbon dioxide, carbon monoxide, water (steam), air, oxygen, and further combinations thereof. The temperature and dwell time of activation can be varied, for example the dwell time can vary from 1 min to 10 min, from 10 min to 30 min, from 30 min to 1 hour, for 1 hour to 2 hours, from 2 hours to 4 hours, from 4 hours to 24 h. The temperature can be varied, for example, the pyrolysis temperature can vary from 200° C. to 300° C., from 250° C. to 350° C., from 350° C. to 450° C., from 450° C. to 550° C., from 540° C. to 650° C., from 650° C. to 750° C., from 750° C. to 850° C., from 850° C. to 950° C., from 950° C. to 1050° C., from 1050° C. to 1150° C., from 1150° C. to 1250° C. In some embodiments, the temperature for combined pyrolysis and activation varies from 650° C. to 1100° C.

In some embodiments, combined pyrolysis and activation is carried out to prepare the porous carbon scaffold. In such embodiments, the process gas can remain the same during processing, or the composition of process gas may be varied during processing. In some embodiments, the addition of an activation gas such as CO2, steam, or combination thereof, is added to the process gas following sufficient temperature and time to allow for pyrolysis of the solid carbon precursors.

Suitable gases for accomplishing carbon activation include, but are not limited to, carbon dioxide, carbon monoxide, water (steam), air, oxygen, and further combinations thereof. The temperature and dwell time of activation can be varied, for example the dwell time can vary from 1 min to 10 min, from 10 min to 30 min, from 30 min to 1 hour, for 1 hour to 2 hours, from 2 hours to 4 hours, from 4 hours to 24 h. The temperature can be varied, for example, the pyrolysis temperature can vary from 200° C. to 300° C., from 250° C. to 350° C., from 350° C. to 450° C., from 450° C. to 550° C., from 540° C. to 650° C., from 650° C. to 750° C., from 750° C. to 850° C., from 850° C. to 950° C., from 950° C. to 1050° C., from 1050° C. to 1150° C., from 1150° C. to 1250° C. In some embodiments, the activation temperature varies from 650° C. to 1100° C.

Either prior to the pyrolysis, and/or after pyrolysis, and/or after activation, the carbon may be subjected to a particle size reduction. The particle size reduction can be accomplished by a variety of techniques known in the art, for example by jet milling in the presence of various gases including air, nitrogen, argon, helium, supercritical steam, and other gases known in the art. Other particle size reduction methods, such as grinding, ball milling, jet milling, water jet milling, and other approaches known in the art are also envisioned.

The porous carbon scaffold may be in the form of particles. The particle size and particle size distribution can be measured by a variety of techniques known in the art, and can be described based on fractional volume. In this regard, the Dv50 of the carbon scaffold may be between 10 nm and 10 mm, for example between 100 nm and 1 mm, for example between 1 μm and 100 μm, for example between 2 μm and 50 μm, example between 3 μm and 30 μm, example between 4 μm and 20 μm, example between 5 μm and 10 μm. In certain embodiments, the Dv50 is less than 1 mm, for example less than 100 μm, for example less than 50 μm, for example less than 30 μm, for example less than 20 μm, for example less than 10 μm, for example less than 8 μm, for example less than 5 μm, for example less than 3 μm, for example less than 1 μm. In certain embodiments, the Dv100 is less than 1 mm, for example less than 100 μm, for example less than 50 μm, for example less than 30 μm, for example less than 20 μm, for example less than 10 μm, for example less than 8 μm, for example less than 5 μm, for example less than 3 μm, for example less than 1 μm. In certain embodiments, the Dv99 is less than 1 mm, for example less than 100 μm, for example less than 50 μm, for example less than 30 μm, for example less than 20 μm, for example less than 10 μm, for example less than 8 μm, for example less than 5 μm, for example less than 3 μm, for example less than 1 μm. In certain embodiments, the Dv90 is less than 1 mm, for example less than 100 μm, for example less than 50 μm, for example less than 30 μm, for example less than 20 μm, for example less than 10 μm, for example less than 8 μm, for example less than 5 μm, for example less than 3 μm, for example less than 1 μm. In certain embodiments, the Dv0 is greater than 10 nm, for example greater than 100 nm, for example greater than 500 nm, for example greater than 1 μm, for example greater than 2 μm, for example greater than 5 μm, for example greater than 10 μm. In certain embodiments, the Dv1 is greater than 10 nm, for example greater than 100 nm, for example greater than 500 nm, for example greater than 1 μm, for example greater than 2 μm, for example greater than 5 μm, for example greater than 10 μm. In certain embodiments, the Dv10 is greater than 10 nm, for example greater than 100 nm, for example greater than 500 nm, for example greater than 1 μm, for example greater than 2 μm, for example greater than 5 μm, for example greater than 10 μm.

In some embodiments, the surface area of the bulk porous carbon scaffold can comprise a surface area greater than 400 m²/g, for example greater than 500 m²/g, for example greater than 750 m²/g, for example greater than 1000 m²/g, for example greater than 1250 m²/g, for example greater than 1500 m²/g, for example greater than 1750 m²/g, for example greater than 2000 m²/g, for example greater than 2500 m²/g, for example greater than 3000 m²/g. In other embodiments, the surface area of the porous carbon scaffold can be less than 500 m²/g. In some embodiments, the surface area of the porous carbon scaffold is between 200 and 500 m²/g. In some embodiments, the surface area of the porous carbon scaffold is between 100 and 200 m²/g. In some embodiments, the surface area of the porous carbon scaffold is between 50 and 100 m²/g. In some embodiments, the surface area of the porous carbon scaffold is between 10 and 50 m²/g. In some embodiments, the surface area of the porous carbon scaffold can be less than 10 m²/g.

In some embodiments, the pore volume of the porous carbon scaffold is greater than 0.4 cm³/g, for example greater than 0.5 cm³/g, for example greater than 0.6 cm³/g, for example greater than 0.7 cm³/g, for example greater than 0.8 cm³/g, for example greater than 0.9 cm³/g, for example greater than 1.0 cm³/g, for example greater than 1.1 cm³/g, for example greater than 1.2 cm³/g, for example greater than 1.4 cm³/g, for example greater than 1.6 cm³/g, for example greater than 1.8 cm³/g, for example greater than 2.0 cm³/g. In other embodiments, the pore volume of the porous carbon scaffold is less than 0.5 cm³, for example between 0.1 cm³/g and 0.5 cm³/g. In certain other embodiments, the pore volume of the porous carbon scaffold is between 0.01 cm³/g and 0.1 cm³/g.

In some other embodiments, the porous carbon scaffold is an amorphous activated carbon with a pore volume between 0.2 and 2.0 cm³/g. In certain embodiments, the carbon is an amorphous activated carbon with a pore volume between 0.4 and 1.5 cm³/g. In certain embodiments, the carbon is an amorphous activated carbon with a pore volume between 0.5 and 1.2 cm³/g. In certain embodiments, the carbon is an amorphous activated carbon with a pore volume between 0.6 and 1.0 cm³/g. In still further embodiments the carbon may be graphite or soft carbon.

In some other embodiments, the porous carbon scaffold comprises a tap density of less than 1.0 g/cm³, for example less than 0.8 g/cm³, for example less than 0.6 g/cm³, for example less than 0.5 g/cm³, for example less than 0.4 g/cm³, for example less than 0.3 g/cm³, for example less than 0.2 g/cm³, for example less than 0.1 g/cm³.

The surface functionality of the porous carbon scaffold can vary. One property which can be predictive of surface functionality is the pH of the porous carbon scaffold. The presently disclosed porous carbon scaffolds comprise pH values ranging from less than 1 to about 14, for example less than 5, from 5 to 8 or greater than 8. In some embodiments, the pH of the porous carbon is less than 4, less than 3, less than 2 or even less than 1. In other embodiments, the pH of the porous carbon is between about 5 and 6, between about 6 and 7, between about 7 and 8 or between 8 and 9 or between 9 and 10. In still other embodiments, the pH is high, and the pH of the porous carbon ranges is greater than 8, greater than 9, greater than 10, greater than 11, greater than 12, or even greater than 13.

The pore volume distribution of the porous carbon scaffold can vary. For example, the % micropores can comprise less than 30%, for example less than 20%, for example less than 10%, for example less than 5%, for example less than 4%, for example less than 3%, for example less than 2%, for example less than 1%, for example less than 0.5%, for example less than 0.2%, for example, less than 0.1%. In certain embodiments, there is no detectable micropore volume in the porous carbon scaffold.

The mesopores comprising the porous carbon scaffold can vary. For example, the % mesopores can comprise less than 30%, for example less than 20%, for example less than 10%, for example less than 5%, for example less than 4%, for example less than 3%, for example less than 2%, for example less than 1%, for example less than 0.5%, for example less than 0.2%, for example, less than 0.1%. In certain embodiments, there is no detectable mesopore volume in the porous carbon scaffold.

In some embodiments, the pore volume distribution of the porous carbon scaffold comprises more than 50% macropores, for example more than 60% macropores, for example more than 70% macropores, for example more than 80% macropores, for example more than 90% macropores, for example more than 95% macropores, for example more than 98% macropores, for example more than 99% macropores, for example more than 99.5% macropores, for example more than 99.9% macropores.

In certain preferred embodiments, the pore volume of the porous carbon scaffold comprises a blend of micropores, mesopores, and macropores. Accordingly, in certain embodiments, the porous carbon scaffold comprises 0-20% micropores, 30-70% mesopores, and less than 10% macropores. In certain other embodiments, the porous carbon scaffold comprises 0-20% micropores, 0-20% mesopores, and 70-95% macropores. In certain other embodiments, the porous carbon scaffold comprises 20-50% micropores, 50-80% mesopores, and 0-10% macropores. In certain other embodiments, the porous carbon scaffold comprises 40-60% micropores, 40-60% mesopores, and 0-10% macropores. In certain other embodiments, the porous carbon scaffold comprises 80-95% micropores, 0-10% mesopores, and 0-10% macropores. In certain other embodiments, the porous carbon scaffold comprises 0-10% micropores, 30-50% mesopores, and 50-70% macropores. In certain other embodiments, the porous carbon scaffold comprises 0-10% micropores, 70-80% mesopores, and 0-20% macropores. In certain other embodiments, the porous carbon scaffold comprises 0-20% micropores, 70-95% mesopores, and 0-10% macropores. In certain other embodiments, the porous carbon scaffold comprises 0-10% micropores, 70-95% mesopores, and 0-20% macropores.

In certain embodiments, the % of pore volume in the porous carbon scaffold representing pores between 100 and 1000 A (10 and 100 nm) comprises greater than 30% of the total pore volume, for example greater than 40% of the total pore volume, for example greater than 50% of the total pore volume, for example greater than 60% of the total pore volume, for example greater than 70% of the total pore volume, for example greater than 80% of the total pore volume, for example greater than 90% of the total pore volume, for example greater than 95% of the total pore volume, for example greater than 98% of the total pore volume, for example greater than 99% of the total pore volume, for example greater than 99.5% of the total pore volume, for example greater than 99.9% of the total pore volume.

In certain embodiments, the pycnometry density of the porous carbon scaffold ranges from about 1 g/cc to about 3 g/cc, for example from about 1.5 g/cc to about 2.3 g/cc. In other embodiments, the skeletal density ranges from about 1.5 cc/g to about 1.6 cc/g, from about 1.6 cc/g to about 1.7 cc/g, from about 1.7 cc/g to about 1.8 cc/g, from about 1.8 cc/g to about 1.9 cc/g, from about 1.9 cc/g to about 2.0 cc/g, from about 2.0 cc/g to about 2.1 cc/g, from about 2.1 cc/g to about 2.2 cc/g or from about 2.2 cc/g to about 2.3 cc/g, from about 2.3 cc to about 2.4 cc/g, for example from about 2.4 cc/g to about 2.5 cc/g.

In some embodiments, the carbon scaffold pore volume distribution can be described as the number or volume distribution of pores as determined as known in the art based on gas sorption analysis, for example nitrogen gas sorption analysis. In some embodiments the pore size distribution can be expressed in terms of the pore size at which a certain fraction of the total pore volume resides at or below. For example, the pore size at which 10% of the pores reside at or below can be expressed as “DPv10”.

The DPv10 for the porous carbon scaffold can vary, for example DPv10 can be between 0.01 nm and 100 nm, for example between 0.1 nm and 100 nm, for example between 1 nm and 100 nm, for example between 1 nm and 50 nm, for example between 1 nm and 40 nm, for example between 1 nm and 30 nm, for example between 1 nm and 10 nm, for example between 1 nm and 5 nm.

The DPv50 for the porous carbon scaffold can vary, for example DPv50 can be between 0.01 nm and 100 nm, for example between 0.1 nm and 100 nm, for example between 1 nm and 100 nm, for example between 1 nm and 50 nm, for example between 1 nm and 40 nm, for example between 1 nm and 30 nm, for example between 1 nm and 10 nm, for example between 1 nm and 5 nm. In other embodiments, the DPv50 is between 2 and 100, for example between 2 and 50, for example between 2 and 30, for example between 2 and 20, for example between 2 and 15, for example between 2 and 10.

The DPv90 for the porous carbon scaffold can vary, for example DPv90 can be between 0.01 nm and 100 nm, for example between 0.1 nm and 100 nm, for example between 1 nm and 100 nm, for example between 1 nm and 50 nm, for example between 1 nm and 50 nm, for example between 1 nm and 40 nm, for example between 1 nm and 30 nm, for example between 1 nm and 10 nm, for example between 1 nm and 5 nm. In other embodiments, the DPv50 is between 2 nm and 100 nm, for example between 2 nm and 50 nm, for example between 2 nm and 30 nm, for example between 2 nm and 20 nm, for example between 2 nm and 15 nm, for example between 2 nm and 10 nm.

In some embodiments, the DPv90 is less than 100 nm, for example less than 50 nm, for example less than 40 nm, for example less than 30 nm, for example less than 20 nm, for example less than 15 nm, for example less than 10 nm. In some embodiments, the carbon scaffold comprises a pore volume with greater than 70% micropores (and DPv90 less than 100 nm, for example DPv90 less than 50 nm, for example DPv90 less than 40 nm, for example DPv90 less than 30 nm, for example DPv90 less than 20 nm, for example DPv90 less than 15 nm, for example DPv90 less than 10 nm, for example DPv90 less than 5 nm, for example DPv90 less than 4 nm, for example DPv90 less than 3 nm. In other embodiments, the carbon scaffold comprises a pore volume with greater than 80% micropores and DPv90 less than 100 nm, for example DPv90 less than 50 nm, for example DPv90 less than 40 nm, for example DPv90 less than 30 nm, for example DPv90 less than 20 nm, for example DPv90 less than 15 nm, for example DPv90 less than 10 nm, for example DPv90 less than 5 nm, for example DPv90 less than 4 nm, for example DPv90 less than 3 nm.

The DPv99 for the porous carbon scaffold can vary, for example DPv99 can be between 0.01 nm and 1000 nm, for example between 0.1 nm and 1000 nm, for example between 1 nm and 500 nm, for example between 1 nm and 200 nm, for example between 1 nm and 150 nm, for example between 1 nm and 100 nm, for example between 1 nm and 50 nm, for example between 1 nm and 20 nm. In other embodiments, the DPv99 is between 2 nm and 500 nm, for example between 2 nm and 200 nm, for example between 2 nm and 150 nm, for example between 2 nm and 100 nm, for example between 2 nm and 50 nm, for example between 2 nm and 20 nm, for example between 2 nm and 15 nm, for example between 2 nm and 10 nm.

In certain embodiments, the carbon scaffold is modified prior to reaction with lithium. For example, in certain embodiments, the surface of the carbon pores is functionalized for the purpose of creating a more lithiophilic surface, i.e., surface that interacts preferentially with lithium or a lithium containing precursor material, wherein said preferential interaction can manifest as preferential diffusion, deposition, or adsorption of the like.

In one embodiment metal oxides are used to functionalize the porous carbon and improve its lithiophilicity. In one instance, a porous carbon scaffold is modified with zinc oxide via a hydrothermal sol-gel synthesis reaction. Zinc acetate dihydrate is dissolved in water and stirred with micronized porous carbon powder. A strong oxidizing agent such as NaOH is then added dropwise into the reaction solution and allowed to react for up to 2 hours, before being separated by filtration and allowed to dry. In some embodiments the metal oxide may be aluminum oxide, nickel oxide, manganese oxide, cobalt oxide, tin oxide, or titanium oxide.

In still further embodiments the metal oxide is deposited via atomic layer deposition or physical vapor deposition onto the porous carbon surface and then subsequently converted to a metal oxide via chemical or thermal oxidation reactions.

In still further embodiments, the porous carbon may be coated with a polymer containing lithium.

### C. Reaction of Lithium in Carbon Via Chemical Vapor Infiltration (CVI)

Chemical vapor deposition (CVD) is a process wherein a substrate provides a solid surface and a gas thermally decomposes on the solid surface of the substrate. A composite may comprise a first component of the solid surface and a second component of the thermally decomposed material on the solid surface. Such a CVD approach can be employed, for instance, to create lithium carbide compounds materials wherein the lithium reacts on the outside surface of a carbon particle. Alternatively, chemical vapor infiltration (CVI) is a process wherein a substrate provides a porous scaffold comprising the first component of the compound, and the gas thermally decomposes within the porosity (into the pores) of the porous scaffold material to react and form a carbide compound within the pores or on an external surface of the porous carbon particle.

In an embodiment, lithium is created within the pores of the porous carbon scaffold by subjecting the porous carbon particles to a lithium containing precursor at elevated temperature such that the lithium containing precursor exists in a gaseous state at the elevated temperature. In certain embodiments, the porous scaffold is a porous pyrolyzed carbon material, and the resulting lithium-pyrolyzed carbon composite created by CVI is subject to processing to activate the carbon material according to activation methods generally described herein. In other embodiments, the porous scaffold is a porous polymer material, and the resulting lithium carbide compound is created by processing to accomplish polymer pyrolysis according to pyrolysis methods generally described herein. The gasified lithium containing precursor can be mixed with other inert gases, for example, nitrogen, argon, and combinations thereof. The temperature and time of processing can be varied, for example the temperature can be between 200° C. and 1700° C., for example between 200° C. and 300° C., for example between 300° C. and 400° C., for example between 400° C. and 500° C., for example between 500° C. and 600° C., for example between 600° C. and 700° C., for example between 700° C. and 800° C., for example between 800° C. and 900° C., for example between 900° C. and 1000° C., for example between 1000° C. and 1100° C., for example between 1100° C. and 1200° C., for example between 1200° C. and 1400° C., for example between 1300° C. and 1400° C. for example between 1400° C. and 1700° C.

In one embodiment, lithium is heated to achieve gasification at or above its boiling point (1330° C.). In other embodiments, the lithium-containing precursor is heated at or above its boiling point to achieve gasification. Exemplary lithium containing precursors in this regard include, but are not limited to, lithium acetylsalicylate (boiling point=350° C.), lithium amide (boiling point=430° C.), lithium bromide (boiling point=1265° C.), lithium tetraborohydride (boiling point=380° C.), lithium chloride (boiling point=1383° C.), lithium hydride (boiling point=950° C.), and lithium hydroxide (boiling point=1626° C.).

The pressure for the CVI process can be varied. In some embodiments, the pressure is atmospheric pressure. In some embodiments, the pressure is below atmospheric pressure. In some embodiments, the pressure is above atmospheric pressure.

### D. Reaction of Lithium in Carbon Via Intrusion

Melt intrusion is a process wherein a liquid infiltrates into the pores of a porous scaffold material. Such a melt intrusion approach can be employed, for instance, to create lithium carbide materials wherein the lithium reacts within the porosity of the porous carbon scaffold material to provide the lithium carbide compound.

Accordingly, the porous scaffold which is subject to melt intrusion can be a porous polymer, porous pyrolyzed carbon, or porous activated carbon. The lithium-polymer composite created by melt intrusion can be subject to subsequent pyrolysis or subject to subsequent pyrolysis and activation to produce the lithium carbide compound. The pressure for the melt intrusion process can be varied. In some embodiments, the pressure is atmospheric pressure. In some embodiments, the pressure is below atmospheric pressure. In some embodiments, the pressure is above atmospheric pressure.

The temperature to accomplish the melt intrusion can vary, for example the temperature can be between 25° C. and 1000° C., for example between 25° C. and 100° C., for example between 100° C. and 200° C., for example between 200° C. and 300° C., for example between 300° C. and 400° C., for example between 400° C. and 500° C., for example between 500° C. and 600° C., for example between 600° C. and 700° C., for example between 700° C. and 800° C., for example between 800° C. and 900° C., for example between 900° C. and 1000° C.

According to one melt intrusion process, the lithium can be in the form of elemental lithium, and the temperature of the process can be varied, for example at or above the melting point of lithium (180.5° C.). In other embodiments, lithium is comprised within a lithium containing precursor, which is heated at or above its melting point to facilitate the melt intrusion process. Exemplary lithium containing precursors in this regard include, but are not limited to, lithium carbonate (melting point=723° C.), lithium acetate (melting point=286° C.), lithium amide (melting point =374° C.), lithium bromide (melting point=550° C.), lithium tetraborohydride (melting point=268° C.), lithium chloride (melting point=610° C.), lithium fluoride (melting point=846° C.), lithium hydride (melting point=689° C.), and lithium hydroxide (melting point=471° C.), lithium hydrogen sulfate (melting point=171° C.), lithium dihydrogen phosphate (melting point=100° C.), lithium nitrate (melting point =261° C.), lithium phosphate (melting point=837° C.), lithium sulfate (melting point=860° C.), lithium sulfide (melting point=950° C.), lithium disulfide (melting point=370° C.), lithium sulfite (melting point=455° C.). Additional exemplary lithium containing precursors include lithium metal alloys including lithium aluminum alloy (melting point=718° C.), lithium aluminum copper alloys (melting point in range of 600° C. to 655° C.), lithium tin alloys (melting point in range of 344° C. to 488° C.), and lithium silicon alloys (melting point=700° C.).

In some embodiments, the non-lithium component of the lithium precursor remains within the lithium carbide compound, and can optionally serve as an electrochemical modifier. In other embodiments, the non-lithium component of the lithium precursor is removed, for example by decomposition, extraction, or other methods known in the art. In other embodiments, any lithium that remains outside of the carbon pores or the porous carbon scaffold can be removed by a solvent wash, where exemplary solvents include, but are not limited to, naphthalene, toluene, or combinations thereof. In some embodiments, the lithium containing precursor introduced into the porous carbon by melt intrusion is converted to lithium by a chemical or electrochemical reduction process before, after, or at the same time it is introduced to the carbon pores.

Exemplary agents for accomplishing reduction of the lithium containing precursor into lithium includes, but are not limited to, hydride reagents and dihydrogen, lithium aluminum hydride, boron hydrides such as sodium borohydride or diborane, metals and organometallic reagents such as the Grignard reagent, and dialkylcopper lithium (lithium dialkylcuprate) reagents such as sodium, alkyl sodium and alkyl lithium.

The melt intrusion process can be carried out in a batch process. Alternatively, the melt intrusion process can be carried out as a continuous process. In some embodiments, the melt intrusion process can be carried out as a continuous process employing extrusion.

In some embodiments, the lithium carbide compound is produced by a melt intrusion process, comprising:
(i) physical mixing of porous carbon scaffold material and a lithium containing precursor material;
(ii) subjecting the mixture to temperature sufficient to achieve melting of the lithium containing precursor;
(iii) intrusion of the molten lithium containing precursor into the pores of the porous carbon scaffold and react with the carbon component.

In other embodiments the lithium carbide compound is produced by a melt intrusion process comprising:
(i) physical mixing of porous carbon scaffold material and a lithium containing precursor material;
(ii) subjecting the mixture to temperature sufficient to achieve melting of the lithium containing precursor;
(iii) intrusion of the molten lithium containing precursor into the pores of the porous carbon scaffold;
(iv) conversion of the lithium containing precursor material into lithium and subsequent reaction with the carbon component.

According to the foregoing embodiment, the conversion of lithium containing precursor into lithium can be accomplished by various methods such as chemical or electrochemical reduction. In certain embodiments, the reduction is accomplished via reaction with a reducing gas environment, such as hydrogen gas.

In some embodiments the lithium carbide compound is produced by a melt intrusion process comprising:
(i) physical mixing of porous carbon scaffold material and a lithium containing precursor material;
(ii) conversion of the lithium containing precursor material into lithium;
(iii) subjecting the mixture to temperature sufficient to achieve melting of the lithium;
(iv) intrusion of the lithium into the pores of the porous carbon scaffold.

According to the foregoing embodiment, the conversion of lithium containing precursor into lithium can be accomplished by various methods such as chemical or electrochemical reduction. In certain embodiments, the reduction is accomplished via reaction with a reducing gas environment, such as hydrogen gas.

In some embodiments the lithium carbide compound is produced by a solute intrusion process comprising:
(i) physical mixing of porous carbon scaffold material and a lithium containing precursor material in the dry state;
(iii) subjecting the mixture to temperature sufficient to achieve melting of the lithium containing precursor;
(iv) subjecting the mixture to temperature and gas sufficient for in situ creation of lithium within the carbon pores;
(v) intrusion of the molten lithium containing precursor into the pores of the porous carbon scaffold and subsequent reaction with the carbon component.

In some embodiments the lithium carbide compound is produced by a solute intrusion process comprising:
(i) physical mixing of porous carbon scaffold material and a lithium containing precursor material dissolved in a solvent;
(ii) removal of the solvent;
(iii) subjecting the mixture to temperature sufficient to achieve melting of the lithium containing precursor;
(iv) intrusion of the molten lithium containing precursor into the pores of the porous carbon scaffold;
(v) conversion of the lithium containing precursor material into lithium.

According to this embodiment, the conversion of lithium containing precursor into lithium can be accomplished by various methods such as chemical or electrochemical reduction. In certain embodiments, the reduction is accomplished via reaction with a reducing gas environment, such as hydrogen gas.

In some embodiments the lithium carbide compound is produced by a solute intrusion process comprising:
(i) physical mixing of porous carbon scaffold material and a lithium containing precursor material dissolved in a solvent;
(ii) removal of the solvent;
(iii) conversion of the lithium containing precursor material into lithium;
(iv) subjecting the mixture to temperature sufficient to achieve melting of the lithium precursor;
(v) intrusion of the molten lithium into the pores of the porous carbon scaffold and subsequent reaction with the carbon component;

According to the foregoing embodiment, the conversion of lithium containing precursor into lithium can be accomplished by various methods such as chemical or electrochemical reduction. In certain embodiments, the reduction is accomplished via reaction with a reducing gas environment, such as hydrogen gas.

### E. Reaction of Lithium and Carbon by Co-Processing Lithium and Carbon Precursors

In some embodiments, carbon and lithium precursors are in situ co-processed to produce the lithium carbide compound. Without being bound by theory, the lithium precursors are incorporated within a polymer resin mixture, wherein the polymer resin mixture is formed as a transient intermediate between precursors and final lithium carbide compound. According to some embodiments, melting of the lithium containing precursor is no greater than the temperature employed to accomplish pyrolysis and/or activation to convert the carbon precursors into carbon. In one such embodiment, the lithium containing precursor can be lithium metal. In other embodiments, the lithium containing precursor can be lithium containing species disclosed elsewhere in this disclosure. Accordingly, the conversion of lithium containing precursor into lithium can be accomplished by various methods such as chemical or electrochemical reduction. In certain embodiments, the reduction is accomplished via reaction with a reducing gas environment, such as hydrogen gas.

Exemplary lithium containing salts useful as precursors include, but are not limited to, dilithium tetrabromonickelate(II), dilithium tetrachlorocuprate(II), lithium azide, lithium benzoate, lithium bromide, lithium carbonate, lithium chloride, lithium cyclohexanebutyrate, lithium fluoride, lithium formate, lithium hexafluoroarsenate(V), lithium hexafluorophosphate, lithium hydroxide, lithium iodate, lithium iodide, lithium metaborate, lithium perchlorate, lithium phosphate, lithium sulfate, lithium tetraborate, lithium tetrachloroaluminate, lithium tetrafluoroborate, lithium thiocyanate, lithium trifluoromethanesulfonate, lithium trifluoromethanesulfonate, lithium hydride, lithium amide, lithium naphthelanide, lithium biphenylide, LiHMDS, and combinations thereof.

### F. Stoichiometry of the Lithium Carbide Compound

The stoichiometry of the lithium carbide compound can vary. For example, in certain embodiments the lithium or carbon content deviates from a 1:1 molar stoichiometry. For instance, the stoichiometry can be in the range of 10 to 0.01, for example 5 to 0.05, for example 2 to 0.1, for example 2 to 0.8, for example 1.5 to 0.8, for example 1.1 to 1.0 molar Li:C ratios. In a preferred embodiment, the stoichiometry ranges from 1.3 to 0.9 molar Li:C ratio. In a preferred embodiment, the stoichiometry ranges from 1.2 to 1.0 molar Li:C ratio. In a preferred embodiment, the stoichiometry ranges from 1.1 to 1.0 molar Li:C ratio.

### G. Reaction of Lithium and Carbon by Electroplating

In one embodiment, the lithium carbide compound can be synthesized via an electroplating mechanism wherein an electrolytic cell is assembled with a porous carbon working electrode (prepared via slurry casting on a copper foil or nickel sheet current collector) and lithium metal counter electrode separated from each other in an liquid electrolyte containing a lithium salt (e.g., LiPF₆, LiFSI, LiTFSI, LiCl, LiBr, LiI, LiNO₃, etc.) and anhydrous organic solvent (e.g., propylene carbonate, ethylene carbonate, 1,3-dioxolane, 1,2-dimethoxyethane, tetrahydrofuran, acetonitrile, etc.). A negative voltage bias (e.g., −1V, −2V, −3V, −4V, −5V, −6V, etc.) is applied to facilitate Li+ reduction in the porous carbon electrode. The amount of charge (Ah) transferred is used to track Li-ion loading and subsequently the applied voltage is stopped once a desired Li loading is achieved. The resulting lithium-carbon electrode is then subjected to an additional thermal reaction step to produce the lithium carbide compound.

An embodiment similar to above wherein the porous carbon electrode is prepared on a roll-to-roll coater that is subsequently conveyed into an electrolyte bath (described above) housed in an inert atmosphere where a negative voltage bias is applied as described in the above embodiment and lithium plating takes place while the electrode is continuously in motion on the rollers. Therefore, the extent of the lithium metal loading is dictated by the conveyance speed of the roll-to-roll apparatus.

Furthermore, the electrolyte bath may contain a dissolved polymer (e.g., polyacrylonitrile, polyvinylidene fluoride, polydopamine, etc.) such that when the electrode leaves the bath and subsequently dries the polymer film is left on the electrode surface acting as a barrier to the atmosphere thus minimizing oxidation of the lithium metal formed in the porous carbon.

An embodiment similar to those above wherein the porous carbon electrode is instead replaced with a carbon nanotube (CNT) scaffold. The CNT's have the advantage of improved electronic conductivity and higher aspect ratios over porous carbon materials and thus could potentially lower the overpotential needed for Li plating on its surface. Furthermore the lack of internal porosity favors a surface centric Li plating/stripping mechanism thereby alleviating resistance due to narrow pore tortuosity.

In a related embodiment, the scaffold comprises a core particle of carbon decorated with carbon structure that are nanosized and/or nano-featured, wherein the decorated moiety serves to promote lithiophilicity. In such embodiments, the core carbon scaffold particle can be porous or non-porous, hard or graphite carbon.

The lithium plating kinetics in the above embodiments can be controlled either galvanostatically (constant current) or potentiostatically (constant voltage). Galvanostatic plating current densities can be controlled from 0.1-0.5, 0.5-1, 1-2, 2-3, 3-4, or 4-5 mA/cm². Sometimes it may be more preferable to instead control the voltage for lithium plating especially when resistances are high and/or when electrode distances are far apart. Some example voltages between the two electrodes may include −0.1 to −0.5, −0.5 to −1, −1 to −2, −2 to −3, −3 to −4, and −4 to −6V. Electrolytes used in these electroplating systems may include one or more lithium salts (e.g., LiPF₆, LiFSI, LiTFSI, LiCl, LiBr, LiI, LiNO₃, LiBOB, LiClO₄ etc.) and concentrations of 0.1-0.5, 0.5-1, 1-2, 2-3, and 3-4 molar. In a solvent consisting of one or more anhydrous organic solvents (e.g., propylene carbonate, ethylene carbonate, diethyl carbonate, fluoroethylene carbonate, vinylidene carbonate, 1,3-dioxolane, 1,2-dimethoxyethane, tetrahydrofuran, acetonitrile, etc.) or ionic liquids (e.g., 1-Butyl-3-methylimidazolium hexafluorophosphate, 1-methyl-1-propylpiperidinium bis(trifluoromethylsulfonyl) imide, N-ethyl-N-methylpyrrolidinium fluorohydrogenate, 1-ethyl-3-methyl-imidazolium bis(fluorosulfonyl)imide).

### H. Coatings Applied to Lithium Carbide Compound

In certain embodiments, the lithium carbide compound particles comprise a terminal particle coating. Without being bound by theory, this coating can impart benefits such as enhanced electrochemical performance and increased safety for materials handling, battery construction and battery operation.

In certain embodiments, the surface layer can comprise a carbon layer. The surface layer is envisioned to provide for a suitable barrier to air and moisture. In this context, the surface carbon layer needs to be a good ionic conductor to shuttle Li-ions. Alternatively, the carbon layer can comprise an artificial SEI layer, for example the carbon layer can comprise poly(3,4-ethylenedioxythiophene)-co-poly(ethylene glycol) copolymer. The coating may comprise nitrogen and/or oxygen functionality to further improve the layer with respect to promoting air stability of the lithium carbide compound. The coating needs to provide sufficient electrical conductivity, adhesion, and cohesion between particles. The surface should provide a stable SEI layer, the latter is typically comprised of species such as LiF, Li₂CO₃, and Li₂O. Inorganic material with relatively low bulk modulus may provide a more stable SEI layer, for example a more amorphous vs. crystalline layer is preferred, for instance Li₂CO₃ vs. LiF.

To this end, a layer of carbon can be applied to the lithium carbide compound particle. Without being bound by theory, this carbon layer should provide low surface area to provide a more stable SEI layer, higher first cycle efficiency, and greater cycle stability in a lithium-ion battery. Various carbon allotropes can be envisioned in the context of providing a surface layer to the lithium carbide materials, including graphite, graphene, hard or soft carbons, for example pyrolytic carbon.

In alternative embodiments, the aforementioned coating can be achieved with a precursor solution as known in the art, followed by a carbonization process. For example, particles can be coated by a Wurster process or related spray drying process known in the art to apply a thin layer of precursor material on the particles. The precursor coating can then be pyrolyzed, for example by further fluidization of the Wurster-coated particles in the presence of elevated temperature and an inert gas as consistent with descriptions disclosed elsewhere herein.

In alternative embodiments, the particles can be covered in a carbonaceous layer accomplished by chemical vapor deposition (CVD). Without wishing to be bound by theory, it is believed that CVD methods to deposit carbon layers (e.g., from a hydrocarbon gas) result in a carbon that is graphitizable (also referred to as “soft” carbon in the art). Methodologies for CVD generally described in the art can be applied to the materials disclosed herein. CVD is generally accomplished by subjecting the compound particulate material for a period of time at elevated temperature in the presence of a suitable deposition gas containing carbon atoms. Suitable gases in this context include, but are not limited to methane, propane, butane, cyclohexane, ethane, propylene, ethylene and acetylene. The temperature can be varied, for example between 350 to 1050° C., for example between 35° and 450° C., for example between 45° and 550° C., for example between 55° and 650° C., for example between 65° and 750° C., for example between 75° and 850° C., for example between 85° and 950° C., for example between 95° and 1050° C. In certain embodiments, the deposition gas is methane, and the deposition temperature is greater than or equal to 950° C. In certain embodiments, the deposition gas is propane, and the deposition temperature is less than or equal to 750° C. In certain embodiments, the deposition gas is cyclohexane and the deposition temperature is greater than or equal to 800° C. In certain embodiments, the deposition gas is acetylene and the deposition temperature is greater than or equal to 400 C. In certain embodiments, the deposition gas is ethylene and the deposition temperature is greater than or equal to 500 C. In certain embodiments, the deposition gas is propylene and the deposition temperature is greater than or equal to 400 C.

In certain embodiments, the reactor to accomplish the coating can be agitated, in order to agitate the lithium carbide compound particles. In other exemplary modes, the particles can be fluidized, for example in a fluidized bed reactor. A variety of different reactor designs can be employed in this context as known in the art, including, but not limited to, elevator kiln, roller hearth kiln, rotary kiln, box kiln, and modified fluidized bed designs.

The thickness of the carbon coating can vary, for example 1-2 nm, 2-5 nm, 5-10 nm, 10-20 nm, 20-50 nm, or 50-100 nm. The mass percentage of the carbon coating on the lithium carbide compound particles as a fraction of the total particle mass can vary, for example 0.01-0.1%, 0.1-0.5%, 0.5-1%, 1-2%, 2-5%, or greater than 5%. In alternative embodiments, the terminal carbon coating can be 0.1% to 5%.

The lithium carbide compound can also comprise a terminal coating that does not comprise carbon. In some embodiments, such a non-carbonaceous coating can be accomplished by atomic layer deposition (ALD) as known in the art. The thickness of the ALD coating can vary, for example 1-2 nm, 2-5 nm, 5-10 nm, 10-20 nm, 20-50 nm, or 50-100 nm. The mass percentage of the ceramic coating on the lithium carbide compound particles as a fraction of the total particle mass can vary, for example 0.01-0.1%, 0.1-0.5%, 0.5-1%, 1-2%, 2-5%, or greater than 5%. Exemplary non-carbonaceous coatings in this regard include, but are not limited to, oxides comprising aluminum, oxides comprising zirconium, and oxides comprising titanium. In alternative embodiments, the terminal ALD coating can be 0.1% to 5%.

I. Doping with Electrochemical Modifiers

In certain embodiments, the lithium carbide compound material can be doped with species that accomplish modification of electrochemical properties. Such electrochemical modifiers can provide enhanced electrochemical properties including, but not limited to, increased capacity, reduced resistance, increased storage stability, and increased cycle stability.

In certain related embodiments, the electrochemical modifier is a metal oxide, for example an oxide of Sn, Ni, In, Ag, Zn, Al, etc, or combinations thereof. In certain related embodiments, the electrochemical modifier comprises a phosphate, for example transition metal phosphate, alkali metal phosphate, or rare earth metal phosphates.

In certain embodiments, the electrochemical modifier can be as a non-metal dopant, for example, oxygen, nitrogen, fluorine, chlorine, phosphorus, silicon, and the like.

### J. Physio- and Electrochemical Properties of Lithium Carbide Compounds and Composites

Certain physicochemical and electrochemical properties of the lithium carbide compound or the lithium carbide composite can vary. Certain such properties are exemplified in Table 1.

TABLE 1
Embodiments for lithium carbide compound or lithium
carbide composite material properties.

| | In some embodiments the lithium |
|---|---|
| | carbide compound of lithium |
| | carbide composite material comprises . . . |
| Surface Area | <100 m²/g, <50 m²/g, <30 m²/g, |
| | <20 m²/g, <10 m²/g, <5 m²/g, |
| | <4 m²/g, <3 m²/g, <2 m²/g, |
| | <1 m²/g, <0.5 m²/g, <0.1 m²/g; |
| First Cycle | >25%, 35>%, >50%, >60%, >70%, >75%, >80%, |
| Efficiency | >85%, >90%, >95%, >97%, >98%, >99%; |
| Reversible | >50 mAh/g, >100 mAh/g, >200 mAh/g, |
| Capacity | >250 mAh/g, >300 mAh/g, >400 mAh/g, |
| | >500 mAh/g, >600 mAh/g, >700 mAh/g, |
| | >800 mAh/g, >900 mAh/g, >1000 mAh/g, |
| | >1200 mAh/g, >1400 mAh/g |

The lithium carbide compound or lithium carbon composite can comprise a combination of the aforementioned properties, and additional properties. Accordingly, Table 2 provides a description of certain embodiments for combination of properties for the lithium carbide compound or the lithium carbide composite.

TABLE 2
Embodiments for lithium carbide compound or lithium
carbide composite material properties.

| | In some embodiments the lithium carbide compound of lithium |
|---|---|
| | carbide composite material comprises . . . |
| Surface Area | <100 m²/g, <50 m²/g, <30 m²/g, <20 m²/g, <10 m²/g, <5 m²/g, |
| | <4 m²/g, <3 m²/g, <2 m²/g, <1 m²/g, <0.5 m²/g, <0.1 m²/g; |
| First Cycle | >25%, 35>%, >50%, >60%, >70%, >75%, >80%, >85%, >90%, |
| Efficiency | >95%, >97%, >98%, >99%; |
| Reversible | >50 mAh/g, >100 mAh/g, >200 mAh/g, >250 mAh/g, >300 |
| Capacity | mAh/g, >400 mAh/g, >500 mAh/g, >600 mAh/g, >700 mAh/g, |
| | >800 mAh/g, >900 mAh/g, >1000 mAh/g, >1200 mAh/g, >1400 mAh/g |
| Average | ≥0.9969, ≥0.9970, ≥0.9975, ≥0.9980, ≥0.9985, ≥0.9990, ≥0.9995, |
| Coulombic | ≥0.9999 |
| efficiency | |
| Pore volume | <0.1 cm³/g, 0.1-0.2 cm³/g, 0.2-0.5 cm³/g, 0.1-1.5 cm³/g, 0.2-1.2 |
| | cm³/g, 0.3-1.1 cm³/g, 0.4-1.0 cm³/g, 0.4-1.0 cm³/g, 0.5-1.0 |
| | cm³/g, 0.6-1.0 cm³/g, 0.5-0.9 cm³/g, 0.4-1.0 cm³/g, >0.1 cm³/g, |
| | >0.2 cm³/g, >0.4 cm³/g, >0.6 cm³/g, >0.8 cm³/g; |
| Pore size range | <1 nm, 1-5 nm, 5-1000 nm, 10-500 nm, 10-200 nm, 10-100 |
| | nm, 33-150 nm, 20-100 nm |
| Percentage of | >20%/>30%/>30%, <10/>30/>30, <5/>30/>30, <5/>40/>40, |
| microporosity/ | <1/>40/>40, <10/>70/>20, <10/>20/>70, >10/>10/>80, |
| mesoporosity/ | <10/>80/>10, <5/>70/>20, <5/>20/>70, <5/>5/>80, |
| macroporosity | <5/>80/>10, >80%/<20%/<20%, >70/<30/<10, >70/<30/<5, |
| expressed as | >70/<20/<10, >70/<10/<10, >70/<10/<5, >70/<5/<5, |
| percentage of | >80/<20/<10, >80/<20/<5, >80/<20/<1, >80/<10/<10, |
| scaffold total | >80/<10/<5, >80/<10/<1, >90/<10/<10, >90/<10/<5, |
| pore volume | >90/<10/<1, >90/<5/<1, >95/<5/<5, >90/<5/<1 |

As used herein, the percentage “microporosity,” “mesoporosity” and “macroporosity” refers to the percent of micropores, mesopores and macropores, respectively, as a percent of total pore volume. For example, a carbon scaffold having 90% microporosity is a carbon scaffold where 90% of the total pore volume of the carbon scaffold is formed by micropores.

Without being bound by theory, the filling and/or creation of lithium carbide within the pores of the porous carbon traps porosity within the porous carbon scaffold particle, resulting in inaccessible volume, for example volume that is inaccessible to nitrogen gas. Accordingly, the lithium carbide material may exhibit a pycnometry density of less than 2.1 g/cm³, for example less than 2.0 g/cm³, for example less than 1.9 g/cm³, for example less than 1.8 g/cm³, for example less than 1.7 g/cm³, for example less than 1.6 g/cm³, for example less than 1.4 g/cm³, for example less than 1.2 g/cm³, for example less than 1.0 g/cm³.

In some embodiments, the lithium carbide material may exhibit a pycnometry density between 1.7 g/cm³ and 2.1 g/cm³, for example between 1.7 g/cm³ and 1.8 g/cm³, between 1.8 g/cm³ and 1.9 g/cm³, for example between 1.9 g/cm³ and 2.0 g/cm³, for example between 2.0 g/cm³ and 2.1 g/cm³. In some embodiments, the lithium carbide material may exhibit a pycnometry density between 1.8 g/cm³ and 2.1 g/cm³. In some embodiments, the lithium carbide material may exhibit a pycnometry density between 1.8 g/cm³ and 2.0 g/cm³. In some embodiments, the lithium carbide compound material may exhibit a pycnometry density between 1.9 g/cm³ and 2.1 g/cm³.

The particle size distribution of the lithium carbide material is important to both determine power performance as well as volumetric capacity. As the packing improves, the volumetric capacity may increase. In one embodiment the distributions are either Gaussian with a single peak in shape, bimodal, or polymodal (>2 distinct peaks, for example trimodal). The properties of particle size of the material can be described by the DO (smallest particle in the distribution), Dv50 (average particle size) and Dv100 (maximum size of the largest particle). The optimal combination of particle packing and performance will be some combination of the size ranges below. The particle size reduction in such embodiments can be carried out as known in the art, for example by jet milling in the presence of various gases including air, nitrogen, argon, helium, supercritical steam, and other gases known in the art.

In one embodiment the Dv0 of the lithium carbide material can range from 1 nm to 5 microns. In another embodiment the Dv0 of the lithium carbide ranges from 5 nm to 1 micron, for example 5-500 nm, for example 5-100 nm, for example 10-50 nm. In another embodiment the Dv0 of the lithium carbide ranges from 500 nm to 2 microns, or 750 nm to 1 μm, or 1-2 μm. microns to 2 microns. In other embodiments, the Dv0 of the lithium carbide ranges from 2-5 μm, or >5 μm.

In some embodiments the Dv50 of the lithium carbide material ranges from 5 nm to 20 μm. In other embodiments the Dv50 of the lithium carbide ranges from 5 nm to 1 μm, for example 5-500 nm, for example 5-100 nm, for example 10-50 nm. In another embodiment the Dv50 of the lithium carbide ranges from 500 nm to 2 μm, 750 nm to 1 μm, 1-2 μm. In still other embodiments, the Dv50 of the lithium carbide ranges from 1 to 1000 μm, for example from 1-100 μm, for example from 1-10 μm, for example 2-20 μm, for example 3-15 μm, for example 4-8 μm. In certain embodiments, the Dv50 is >20 μm, for example >50 μm, for example >100 μm.

The span (Dv50)/(Dv90-Dv10), wherein Dv10, Dv50 and Dv90 represent the particle size at 10%, 50%, and 90% of the volume distribution, can be varied from example from 100 to 10, from 10 to 5, from 5 to 2, from 2 to 1; in some embodiments the span can be less than 1. In certain embodiments, the lithium carbide material particle size distribution is unimodal. In certain embodiments, the lithium carbide material particle size distribution has a right hand skew. In certain embodiments, the lithium carbide material particle size distribution has a left hand skew. In certain embodiments, the lithium carbide material particle size distribution can be multimodal, for example, bimodal, or trimodal.

The surface functionality of the presently disclosed lithium carbide material may be altered to obtain the desired electrochemical properties. One property which can be predictive of surface functionality is the pH of the material. The presently disclosed lithium carbide materials comprise pH values ranging from less than 1 to about 14, for example less than 5, from 5 to 8 or greater than 8. In some embodiments, the pH of the lithium carbide materials is less than 4, less than 3, less than 2 or even less than 1. In other embodiments, the pH of the lithium carbide materials is between about 5 and 6, between about 6 and 7, between about 7 and 8 or between 8 and 9 or between 9 and 10. In still other embodiments, the pH is high and the pH of the materials range is greater than 8, greater than 9, greater than 10, greater than 11, greater than 12, or even greater than 13.

The lithium carbide material may comprise varying amounts of oxygen, hydrogen, and nitrogen as measured by CHNO analysis. In some embodiments, lithium carbide compound material comprises a nitrogen content ranging from 0-90%, for example 0.1-1%, for example 1-3%, for example 1-5%, for example 1-10%, for example 10-20%, for example 20-30%, for example 30-90%.

In some embodiments, the oxygen content ranges from 0-90%, for example 0.1-1%, for example 1-3%, for example 1-5%, for example 1-10%, for example 10-20%, for example 20-30%, for example 30-90%.

The morphology of the lithium carbide compounds can vary. For example, the lithium carbide particles are spherical in shape.

The lithium carbide compound material may also incorporate an electrochemical modifier selected to optimize the electrochemical performance of the non-modified compound. The electrochemical modifier may be incorporated within the pore structure and/or on the surface of the porous carbon scaffold, within the embedded lithium carbide, or within the final layer of carbon, or conductive polymer, coating, or incorporated in any number of other ways. For example, in some embodiments, the lithium carbide materials comprise a coating of the electrochemical modifier (e.g., lithium or Al₂O₃) on the surface. In some embodiments, the lithium carbide materials comprise greater than about 100 ppm of an electrochemical modifier. In certain embodiments, the electrochemical modifier is selected from iron, tin, silicon, nickel, aluminum and manganese.

The electrochemical modifier may be provided in any number of forms. For example, in some embodiments the electrochemical modifier comprises a salt. In other embodiments, the electrochemical modifier comprises one or more elements in elemental form, for example elemental iron, tin, silicon, nickel or manganese. In other embodiments, the electrochemical modifier comprises one or more elements in oxidized form, for example iron oxides, tin oxides, silicon oxides, nickel oxides, aluminum oxides or manganese oxides.

In certain embodiments, the lithium carbide material is tested in a half-cell; alternatively, the performance of the material is tested in a full cell, for example a full cell coin cell, a full cell pouch cell, a prismatic cell, or other battery configurations known in the art. The cathode composition comprising the compound can further comprise various species, as known in the art. Additional formulation components include, but are not limited to, conductive additives, such as conductive carbons such as Super C45, Super P, Ketjenblack carbons, and the like, conductive polymers and the like, binders such as styrene-butadiene rubber sodium carboxymethylcellulose (SBR-Na-CMC), polyvinylidene difluoride (PVDF), polyimide (PI), polyacrylic acid (PAA) and the like, and combinations thereof. In certain embodiments, the binder can comprise a lithium ion as a counterion (e.g., lithium polyacrylic acid (LiPAA), lithium carboxymethylcellulose (Li-CMC), etc.).

Other species comprising the electrode are known in the art. The % of active material in the electrode by weight can vary, for example between 1 and 5%, for example between 5 and 15%, for example between 15 and 25%, for example between 25 and 35%, for example between 35 and 45%, for example between 45 and 55%, for example between 55 and 65%, for example between 65 and 75%, for example between 75 and 85%, for example between 85 and 95%. In some embodiments, the active material comprises between 80 and 95% of the electrode. In certain embodiments, the amount of conductive additive in the electrode can vary, for example between 1 and 5%, between 5 and 15%, for example between 15 and 25%, for example between 25 and 35%. In some embodiments, the amount of conductive additive in the electrode is between 5 and 25%. In certain embodiments, the amount of binder can vary, for example between 1 and 5%, between 5 and 15%, for example between 15 and 25%, for example between 25 and 35%. In certain embodiments, the amount of conductive additive in the electrode is between 5 and 25%.

The cathode comprising the lithium carbide material can be paired with various anode materials to result in a full cell lithium-ion battery. Examples of suitable anode materials are known in the art. Examples of such anode materials include, but are not limited to, graphite, silicon, and silicon-carbon composite materials. Exemplary silicon-carbon composite materials are described in U.S. Pat. Nos. 10,147,950 and 11,174,167, and application/publication documents U.S. Ser. No. 18/042,173, U.S. Ser. No. 18/246,472, PCT/US2022/036127, and PCT/US2021/052995 all of the foregoing are herein incorporated by reference in their entirety.

For the full cell comprising a lithium carbide cathode, the pairing of cathode to anode can be varied. For example, the ratio of cathode-to-anode capacity can vary from 0.7 to 1.3. In certain embodiments, the ratio of cathode-to-anode capacity can vary from 0.7 to 1.0, for example from 0.8 to 1.0, for example from 0.85 to 1.0, for example from 0.9 to 1.0, for example from 0.95 to 1.0. In other embodiments, the ratio of cathode-to-anode capacity can vary from 1.0 to 1.3, for example from 1.0 to 1.2, for example from 1.0 to 1.15, for example from 1.0 to 1.1, for example from 1.0 to 1.05. In yet other embodiments, the ratio of cathode-to-anode capacity can vary from 0.8 to 1.2, for example from 0.9 to 1.1, for example from 0.95 to 1.05.

For the full cell battery comprising the lithium carbide cathode, the voltage window for charging and discharging can be varied. In this regard, the voltage window can be varied as known in the art. For instance, the choice of cathode plays a role in the voltage window chosen, as known in the art. Examples of voltage windows vary, for example, in terms of potential versus Li/Li+, from 2.0 V to 5.0 V, for example from 2.5 V to 4.5V, for example from 2.5V to 4.2V. For the full cell battery comprising the lithium carbide cathode, the strategy for conditioning the cell can be varied as known in the art. For example, the conditioning can be accomplished by one or more charge and discharge cycles at various rate(s), for example at rates slower than the desired cycling rate. As known in the art, the conditioning process may also include a step to unseal the lithium ion battery, evacuate any gases generated during the conditioning process, followed by resealing the lithium ion battery.

For the battery comprising the lithium carbide cathode, the cycling rate can be varied as known in the art, for example, the rate can be between C/20 and 20 C, for example between C10 to 10 C, for example between C/5 and 5 C. In certain embodiments, the cycling rate is C/10. In certain embodiments, the cycling rate is C/5. In certain embodiments, the cycling rate is C/2. In certain embodiments, the cycling rate is 1 C. In certain embodiments, the cycling rate is 1 C, with periodic reductions in the rate to a slower rate, for example cycling at 1 C with a C/10 rate employed every 20^{th} cycle. In certain embodiments, the cycling rate is 2 C. In certain embodiments, the cycling rate is 4 C. In certain embodiments, the cycling rate is 5 C. In certain embodiments, the cycling rate is 10 C. In certain embodiments, the cycling rate is 20 C.

The full cell battery comprising lithium carbide cathode exhibits a first cycle efficiency (FCE). In preferred embodiments, the FCE is greater or equal to 70%, for example 80%, for example 85%, for example 90%, for example 95%, for example 96%, for example 98%, for example 99%.

In certain embodiments, the electrolyte can comprise various additives known to provide improved performance, such as fluoroethylene carbonate (FEC) or other related fluorinated carbonate compounds, or ester co-solvents such as methyl butyrate, vinylene carbonate, and other electrolyte additives known to improve electrochemical performance.

Coulombic efficiency can be averaged, for example averaged over cycles 2 or later to cycle 20 or later when tested in a half cell. In certain embodiments, the average efficiency of the lithium carbide is greater than 0.9, or 90%. In certain embodiments, the average efficiency is greater than 0.95, or 95%. In certain other embodiments, the average efficiency is 0.99 or greater, for example 0.991 or greater, for example 0.992 or greater, for example 0.993 or greater, for example 0.994 or greater, for example 0.995 or greater, for example 0.996 or greater, for example 0.997 or greater, for example 0.998 or greater, for example 0.999 or greater, for example 0.9991 or greater, for example 0.9992 or greater, for example 0.9993 or greater, for example 0.9994 or greater, for example 0.9995 or greater, for example 0.9996 or greater, for example 0.9997 or greater, for example 0.9998 or greater, for example 0.9999 or greater.

### EXAMPLES

### Example 1. Properties of Various Carbon Scaffold Materials

The properties of various carbon scaffold materials are presented in Table 3. The exemplary carbon materials vary in properties such as total pore volume (for example varying from 0.5 to greater than 2 cm³/g, and also varying percentages of micro-, meso- and macropores.

TABLE 3
Properties of various carbon scaffold materials.

| Carbon | Carbon | Surface | Pore | % | % | % |
|---|---|---|---|---|---|---|
| Scaffold | Phase | Area | Volume | Micro- | Meso- | Macro- |
| # | Type | (m²/g) | (cm³/g) | pores | pores | pores |
| 1 | Amorphous | 1452 | 0.652 | 88.4 | 11.4 | 0.2 |
| 2 | Amorphous | 1259 | 0.548 | 89.3 | 10.4 | 0.3 |
| 3 | Amorphous | 1128 | 0.595 | 77.4 | 22.5 | 0.1 |
| 4 | Crystalline | 18.6 | 0.046 | 4.1 | 72.8 | 23.1 |
| 5 | Amorphous | 1459 | 0.649 | 84.6 | 15.1 | 0.3 |
| 6 | Nanocrystalline | 45 | 0.088 | 7 | 71.8 | 21.2 |
| 7 | Amorphous | 4.9 | 0.005 | 15.5 | 61.4 | 23.1 |
| 8 | Amorphous | Pending | Pending | Pending | Pending | Pending |
| 9 | Amorphous | 1744 | 0.721 | 94.3 | 2.8 | 2.9 |

^{a}Carbon scaffold was monolithic, all other carbon scaffolds were particulate.

### Example 2. Melt Infusion Method of Synthesis for Lithium Carbide Compound (LCC)

In a typical but preferred embodiment a portion of micronized porous carbon powder is placed in a metal or ceramic crucible and physically mixed with a portion of lithium metal in the form of foil or powder. The Li:C weight ratio is adjusted so as to partially fill the available pore volume of the carbon allowing for some residual void (e.g., 0.1, 0.2, 0.3, 0.4, 0.5, 0.6, 0.7, 0.8, 0.9, or 1 w/w Li:C). The mixture is then heated under in an inert atmosphere (e.g., argon, nitrogen, helium, or vacuum) to at least the melting point of the lithium metal (e.g, 180° C., 190° C., 200° C., 220° C., 250° C., 300° C., 400° C., etc.). The mixture dwells at peak temperature for a period of time (e.g., 0.1 hr, 1 hr, 2 hr, 5 hr, 10 hr, 24 hr, etc.) to allow molten lithium to permeate the carbon pore structure via capillary forces. The LCC is formed at this time then subsequently cooled to ambient temperature and removed for processing.

In another embodiment the lithium metal and porous carbon powder are kept separated in the same heated reactor environment and the temperature is heated much hotter to increase the vapor pressure of the molten lithium (e.g., 900° C., 1000° C., 1100° C., 1200° C., 1300° C., 1350° C., etc.). This would facilitate vapor phase deposition of lithium metal within the pore structure of the carbon via capillary condensation. The Li:C ratio would therefore be controlled by the dwell time at peak temperature (e.g., 0.1 hr, 1 hr, 2 hr, 5 hr, 10 hr, 24 hr, etc.).

In yet another embodiment the lithium metal source is in the form of an electrode/target for a plasma physical vapor deposition apparatus and the porous carbon is acting as the counter electrode. The synthesis of the LCC is performed by applying a voltage bias between the electrodes under a partial pressure of argon gas. This facilitates evaporation of the lithium metal via ion bombardment resulting in lithium metal deposition taking place on the porous carbon. The rate of deposition can be controlled by the applied voltage bias and current. The Li:C ratio can be controlled by dwell time similar to the above embodiments.

### Example 3. Liquid Phase Methods of Synthesis for Lithium Carbide Compounds

In a typical embodiment a solution of naphthalene in an anhydrous aprotic ethereal solvent (e.g., tetrahydrofuran (THF), dimethoxyethane (DME), diethyl ether etc.) is prepared in an inert gas environment (e.g., argon, nitrogen, helium, etc.). While stirring or sonicating a portion of lithium metal (1:1 molar ratio to naphthalene) is added to the solution in the form of foil, pellets, or powder. The lithium metal is allowed to completely dissolve to a transparent green solution. Porous carbon is then added to the solution in a desired Li:C ratio as indicated in Example 1. Subsequently the solvent and naphthalene are then removed from the mixture via either solvent exchange with a non-ethereal aprotic solvent (e.g., toluene, acetonitrile, etc.) followed by evaporation to yield the dry LCC material which can then be removed for processing.

In another perhaps preferred embodiment the same synthesis procedure as in Example 1 is conducted but the mixture is then heated to a temperature so as to facilitate evaporation of both the naphthalene and solvent species (e.g., >220° C.). Leaving behind only the LCC material and foregoes the use of additional solvents.

### Example 4. Vapor Phase Methods of Synthesis for Lithium Carbide Compounds

An embodiment wherein lithium is created within the pores of the porous carbon scaffold by subjecting the porous carbon particles to a lithium containing precursor gas via chemical vapor infiltration (CVI) at elevated temperature and the presence of a lithium-containing gas, preferably lithium bis(trimethylsilyl)amide, in order to decompose said gas into lithium. In some embodiments, the lithium containing gas may be composed of organic derivatives (such as methyl lithium, phenyl lithium, and the like) or mixtures thereof. The lithium containing precursor gas can be mixed with other inert gas(es), for example, nitrogen gas, or hydrogen gas, or argon gas, or helium gas, or combinations thereof. The temperature and time of processing can be varied, for example the temperature can be between 100° C. and 900° C., for example between 100° C. and 250° C., for example between 250° C. and 300° C., for example between 300° C. and 350° C., for example between 300° C. and 400° C., for example between 350° C. and 450° C., for example between 350° C. and 400° C., for example between 400° C. and 500° C., for example between 500° C. and 600° C., for example between 600° C. and 700° C., for example between 700° C. and 800° C., for example between 800° C. and 900° C., for example between 600° C. and 1100° C. The mixture of gas can comprise between 0.1 and 1% gaseous lithium precursor and remainder inert gas. Alternatively, the mixture of gas can comprise between 1% and 10% lithium precursor and remainder inert gas. Alternatively, the mixture of gas can comprise between 10% and 20% lithium precursor and remainder inert gas. Alternatively, the mixture of gas can comprise between 20% and 50% lithium precursor and remainder inert gas. Alternatively, the mixture of gas can comprise above 50% lithium precursor and the remaining inert gas. Alternatively, the gas can essentially be 100% lithium precursor gas. The pressure for the CVI process can be varied. In some embodiments, the pressure is atmospheric pressure. In some embodiments, the pressure is below atmospheric pressure. In some embodiments, the pressure is above atmospheric pressure.

### Example 5. Addition of Alloying Species for Synthesis of Lithium Carbide Compounds

As is known in the art, lithium metal can be alloyed with other elements in some cases forming lower melting point (<180° C.) eutectic mixtures. These eutectic mixtures can be exploited to more easily direct formation/precipitation of lithium metal within the porous carbon structure. In one such embodiment, the porous carbon scaffold is first loaded with an alloying agent (e.g., silver) in the form of a solution containing the alloy precursor (e.g., 0.1M silver nitrate in water). The solution is added to the dry porous carbon powder via a technique known in the art as incipient wetness at a low relative concentration (e.g., 0.1%, 1%, 2%, 5%, or 10% w/w Ag:C). The water solvent is subsequently removed via evaporation and the alloy precursor is decomposed/reduced to its metal neutral oxidation state (i.e., silver metal) throughout the pore structure of the carbon in the form of discrete nanoparticles (e.g., 1-50 nm in diameter). This Ag/C composite can then be used as the host material for lithium metal formation as described in the above synthesis Examples. In the case of Example 1, the melt infusion step of lithium metal within the carbon pores would preferentially occur where there is a silver nanoparticle since the eutectic melting point of ˜0.1 w/w Li/Ag alloy occurs at a lower temperature than lithium metal itself (i.e., 143° C. versus 180° C. for pure lithium). As the eutectic Li/Ag alloy reaches a lithium saturation point it will precipitate solid lithium from the eutectic melt thus directing the bulk of lithium metal formation in the carbon pore structure where the silver nanoparticles originally resided. In another embodiment as in the case of Example 3, the silver nanoparticles within the carbon pore structure can act as a catalytic seed particle for deposition and subsequent alloying of lithium metal from the lithium precursor gas during CVI.

### Example 6. Reduction of Lithium Salts for Synthesis of Lithium Carbide Compounds

An embodiment wherein lithium is created within the pores of the porous carbon scaffold by mixing the porous carbon particles with lithium salt (e.g., LiF, LiCl, LiNO₃, Li₂CO₃, LiI, LiBr, LiAlH₄, LiOH, LizO, LiO₂, Li₃N, etc.) at elevated temperature with or without the presence of a reducing agent (e.g., H2, NaBH₄, oxalic acid, glucose, carbon, etc.) in order to decompose said salt into lithium metal. The lithium salt can be pre-dissolved in solvents (e.g., tetrahydrofuran, propylene carbonate, acetone, etc.) so as to more easily flow/absorb into the nano-pores of the porous carbon scaffold. The reduction temperature and time of processing can be varied, for example the temperature can be between 0° C. and 900° C., for example between 0° C. and 250° C., for example between 250° C. and 300° C., for example between 300° C. and 350° C., for example between 300° C. and 400° C., for example between 350° C. and 450° C., for example between 350° C. and 400° C., for example between 400° C. and 500° C., for example between 500° C. and 600° C., for example between 600° C. and 700° C., for example between 700° C. and 800° C., for example between 800° C. and 900° C., for example between 600° C. and 1100° C. The solvent/salt mixture can comprise between 0.1 and 1% lithium salt and remainder liquid solvent. Alternatively, the mixture of solvent/salt can comprise between 1% and 10% lithium salt and remaining liquid solvent. Alternatively, the mixture of solvent/salt can comprise between 10% and 20% lithium salt and remaining liquid solvent. Alternatively, the mixture of solvent/salt can comprise between 20% and 50% lithium salt and remainder liquid solvent. Alternatively, the mixture of solvent/salt can comprise above 50% lithium salt and remaining liquid solvent. Alternatively, the solvent/salt can essentially be 100% lithium salt. The pressure for the reduction process can be varied. In some embodiments, the pressure is atmospheric pressure. In some embodiments, the pressure is below atmospheric pressure. In some embodiments, the pressure is above atmospheric pressure.

### Example 7. Electrochemical Methods of Forming Lithium Carbide Compounds

In one embodiment, the lithium carbide compound can be synthesized via an electroplating mechanism wherein an electrolytic cell is assembled with a porous carbon working electrode (prepared via slurry casting on a copper foil or nickel sheet current collector) and lithium metal counter electrode separated from each other in an liquid electrolyte containing a lithium salt (e.g., LiPF₆, LiFSI, LiTFSI, LiCl, LiBr, LiI, LiNO₃, etc.) and anhydrous organic solvent (e.g., propylene carbonate, ethylene carbonate, 1,3-dioxolane, 1,2-dimethoxyethane, tetrahydrofuran, acetonitrile, etc.). A negative voltage bias (e.g., −1V, −2V, −3V, −4V, −5V, −6V, etc.) is applied to facilitate Li+ reduction in the porous carbon electrode. The amount of charge (Ah) transferred is used to track Li metal loading and subsequently the applied voltage is stopped once a desired Li loading is achieved. The lithium-carbon electrode can then be transferred to and used as the cathode in a Li-ion battery.

An embodiment similar to above wherein the porous carbon electrode is prepared on a roll-to-roll coater that is subsequently conveyed into an electrolyte bath (described above) housed in an inert atmosphere where a negative voltage bias is applied as described in the above embodiment and lithium plating takes place while the electrode is continuously in motion on the rollers. Therefore, the extent of the lithium metal loading is dictated by the conveyance speed of the roll-to-roll apparatus.

Furthermore, the electrolyte bath may contain a dissolved polymer (e.g., polyacrylonitrile, polyvinylidene fluoride, polydopamine, etc.) such that when the electrode leaves the bath and subsequently dries the polymer film is left on the electrode surface acting as a barrier to the atmosphere thus minimizing oxidation of the lithium metal formed in the porous carbon.

In an alternative more preferred embodiment the lithium electroplating can be performed in-situ in an as-assembled Li-ion battery wherein the porous carbon electrode (described above) is the anode and a conventional Li-bearing transition metal oxide as known in the art (e.g., LiFePO₄, LiCoO₂, NCA, NMC111, NMC532, NMC622, etc.) acts as the cathode. Lithium electroplating takes place as the battery is charged to its 100% state of charge operating voltage (e.g., 4.2V). In this “anode-free” configuration the Li+ source is the cathode. The process is reversed (Li+ stripping from the porous carbon electrode) when the battery is discharged. This embodiment is preferred because it does not require reactive lithium metal to be handled in an environment outside the battery and furthermore the energy density of the battery can be improved since the cathode acts as the sole source of Li⁺ in the system.

### Example 8. Terminal Coating Methods for Lithium Carbide Compounds

Owing to the highly reactive nature of lithium carbide in atmospheric conditions (e.g., oxidative reaction with water, oxygen, and carbon dioxide) it may be necessary to coat/protect the surface of the lithium carbide compound utilizing terminal coating methods described herein. In one embodiment following synthesis of the lithium carbide compound as described in Examples 1-6 the compound is subsequently heated to temperature (e.g., 400-1000° C.) so as to facilitate decomposition of a hydrocarbon gas (e.g., acetylene, propylene, ethylene, methane, propane, propadiene/propyne, etc.). At peak temperature the hydrocarbon gas is introduced into the heated chamber containing the lithium carbide compound material and allowed to undergo a chemical vapor deposition reaction depositing carbon on the surface of the lithium carbide compound material according to the reaction equation CₓH_{y}->C+H2. The thickness of the coating can be controlled by the dwell time in which the hydrocarbon gas is present (e.g., 0.1 hr-6 hr). The application of the carbon coating will subsequently protect the silicon from oxidation in atmospheric conditions. In another embodiment the lithium carbide compound material can be coated with a polymer (e.g., polydopamine, polyacrylonitrile, polyaniline, polypyrrole, etc.) to allow for lower temperature (e.g., <200° C.) processing.

In some embodiments, lithium carbide compound particles can be coated with carbon to improve a particular physical property (e.g., electrical conductivity) or performance attribute (e.g., electrochemical cycle stability). In a typical example, lithium carbide compound powder is weighed out (˜1 gram) and added to an inconel crucible then introduced into a horizontal tube furnace. The furnace atmosphere is inerted with either a noble gas (e.g., argon) or vacuum then heated to 400° C. At peak temperature, a hydrocarbon gas such as acetylene is introduced at a nominal flow rate (˜0.5 SLPM). The temperature and flow rate are kept constant for a specified dwell time (˜30 minutes) to allow for chemical vapor deposition of amorphous carbon to take place on the lithium carbide compound surface. After dwell, the furnace atmosphere is once again inerted and subsequently cooled to room temperature wherein the sample is removed into a glovebox and harvested for analysis.

In some embodiments, the hydrocarbon gas used for carbon coating the lithium carbide compound particles is methane, propane, ethane, butane, butylene, benzene, toluene, styrene, propylene, or acetylene, or combinations thereof.

In some embodiments, the carbon coating of the lithium carbide material is at a dwell temperature is between 200° C. and 1000° C., or 200° C. and 400° C., or 400° C. and 500° C., or 500° C. and 600° C., or 600° C. and 700° C., or 700° C. and 800° C., or 800° C. and 1000° C. In a preferred embodiment, the dwell temperature is 200° C. to 450° C. to avoid melting of the lithium carbide compound. In some embodiments, the carbon coating of the lithium carbide compound material is at a dwell time between 0.1 and 12 hours, for example 0.1 to 1 h, for example 1 h and 2 h, for example 2h to 4 h, for example 4 h to 12 h. In some embodiments, the hydrocarbon gas is diluted with a carrier gas, and exemplary carrier gases include, but are not limited to, nitrogen, argon, helium, hydrogen, and combinations thereof. In some embodiments, the carbon coating process is carried out at atmospheric pressure. In other embodiments, the carbon coating process is carried above atmospheric pressure. In other embodiments, the carbon coating process is carried out below atmospheric pressure.

### Example 9. Surface Functionality Methods and Metrics

The surface functionality of the presently disclosed lithium carbide compound material comprising carbon and lithium may be altered to obtain the desired electrochemical properties. One such property for particulate lithium carbide materials is the concentration of atomic species at the surface of the material relative to the interior of the material. Such a difference in concentration of atomic species of the surface vs. interior of the particulate material can be determined as known in the art, for example by x-ray photoelectron spectroscopy (XPS). For example, the concentration of Li:C at the surface (defined as the terminal 5 nm of the particulate surface) may be determined by this method. In some embodiments the ratio of Li:C at the surface ranges from about 0.1:1 to 10:1. In certain other embodiments, the ratio of Li:C at the surface is about 0:1. In other embodiments, the ratio of Li:C at the surface is about 1:1. In another example, the Li:O ratio at the surface ranges from about 0:1 to 1:0.

Another property which can be predictive of surface functionality is the pH of the lithium carbide compounds. The presently disclosed materials comprise pH values ranging from less than 1 to about 14, for example less than 5, from 5 to 8 or greater than 8. In some embodiments, the pH of the lithium carbide materials is less than 4, less than 3, less than 2 or even less than 1. In other embodiments, the pH of the lithium carbide materials is between about 5 and 6, between about 6 and 7, between about 7 and 8 or between 8 and 9 or between 9 and 10. In still other embodiments, the pH is high and the pH of the materials ranges is greater than 8, greater than 9, greater than 10, greater than 11, greater than 12, or even greater than 13.

Other methods and metrics for determination of lithium carbide materials structure include X-ray diffraction (XRD) and Raman spectroscopic analysis. With regards to XRD, the graphitic nature of carbon materials can be assessed by monitoring peak intensity at various 2q corresponding to various Miller indices. Without being bound by theory, diffraction lines of graphite are classified into various groups, such as 001, hk0, and hkl indices, mainly because of the strong anisotropy in structure. One such species is 002, corresponding to basal planes of graphite, which is located at 20-26°; this peak is prominent in highly graphitic carbon materials. Carbon material with lesser extent of graphite nature and small crystallite sizes may be characterized by very broad 001 lines (e.g., 002) and shifting (e.g. 2θ˜23°), due to the lesser extent of stacked layers, and by unsymmetrical hk lines (e.g., 10 corresponding to 2θ˜43°). Furthermore, the Scherrer formula may be used to calculate crystallite size (Lc) from the 002 line and crystallite size (La) from the 100 line.

With regards to Raman spectroscopy, this method can also be employed to assess graphite nature of carbon as reported in the art The position, shape, and magnitude of the Raman D- and G bands is known to the art for calculation of the La value from the Tuinstra Koenig (TK) model for >2 nm grain size or the Ferrari (FR) model (Ferrari, A. C., & Robertson, J. (1970); Tuinstra, F., & Koening, J. L. (1970). Raman spectrum of graphite. The Journal of Chemical Physics, 53(3), 1126-1130). Interpretation of Raman spectra of disordered and amorphous carbon. Physical Review B, 61(20), 14095-14107) when TK model calculates <2 nm grain size. These models provide a measure of the disorder in carbon materials and represent the length of the graphene crystallite sheets in carbon materials.

Yet another analysis method is determination of oxygen, nitrogen and hydrogen employing an inert gas fusion instrument. The lithium carbide compound material may comprise varying amounts of carbon, oxygen, hydrogen and nitrogen as measured by an inert gas fusion instrument known in the art (LECO ONH 836). The lithium carbide compound sample is flash heated in a graphite arc furnace to ˜3000° C. under flowing helium gas. The oxygen in the sample is carbo-thermally reduced to CO2 and/or CO which is entrained in the helium gas stream and quantified downstream using an IR spectrometer. Hydrogen is evolved from the sample in the form of H₂ which is converted catalytically to H2O in the gas phase and quantified also using an IR spectrometer. Lastly, the nitrogen is evolved from the sample in the form of N2 and quantified using a thermal conductivity detector.

In some embodiments, lithium carbide compound material comprises a nitrogen content ranging from 0-90%, for example 0.1-1%, for example 1-3%, for example 1-5%, for example 1-10%, for example 10-20%, for example 20-30%, for example 30-90%. In some embodiments, the oxygen content ranges from 0-90%, for example 0.1-1%, for example 1-3%, for example 1-5%, for example 1-10%, for example 10-20%, for example 20-30%, for example 30-90%.

### Example 10. Stability of Lithium Carbide Compound Under Ambient Conditions

The instability of lithium metal under ambient conditions is well known in the art. The current disclosure provides for a lithium that is protected within a porous carbon scaffold, with optional terminal coating applied to the particle. This protection can be described in terms of the confinement of lithium within the carbon scaffold and is manifested as decreased or eliminated reactivity in air (oxygen), stability in contact with other battery components (chemical), stability in operation (electrochemical), and suppression of dendrites upon battery cycling. For example, a metric such as onset time or severity for reaction with organic solvent can be measured by H₂ evolution and/or total quantity. Alternatively, one can measure onset time or severity of tarnishing/color change/oxidation of lithium-carbon in air. Alternatively, stability can be assessed by TGA/DSC by measuring mass uptake due to oxidation of the lithium within the compound. In addition, DSC also is known to provide information about lithium melting point, whose alteration yields information about the stability and/or disposition of lithium within the carbon scaffold porosity. Alternatively, stability can be measured in a half cell vs. lithium metal to determine the number of galvanostatic cycles until dendrite failure, i.e., short circuit of the half cell. Alternatively, stability can be assessed by small angle X-ray scattering (SAXS) or neutron scattering to determine the distribution and size of lithium in the pore of the porous carbon.

### Example 11. Lithium Carbide Synthesis Employing Lithium Hydride

In some embodiments, lithium carbide compound is prepared by weighing out equimolar amounts of lithium hydride and carbon in an inert environment consisting of either a noble gas (e.g., argon) or vacuum. The carbon surface can be modified using various reactive gas treatments: hydrogen, carbon dioxide, nitrogen, to enrich the surface composition in their respective elemental species. The powders are mixed until homogenous in color with a mortar and pestle and loaded into an inconel crucible. The material is then heated under an inert argon gas flow at a nominal flow rate (˜0.5 SLPM) in a horizontal tube furnace to a peak dwell temperature (900° C.) and allowed to dwell for a period of time (2 hours) for the reaction to complete, forming a pure lithium carbide phase. The crucible is then allowed to passively cool under inert atmosphere until room temperature is reached. The sample is then collected and micronized via attrition milling to a particle size below 25 microns for characterization and electrochemical testing.

In some embodiments, the lithium carbide synthesized employing lithium hydride comprises a carbon structure is graphitic, nano-crystalline, or amorphous. In some embodiments, the carbon form factor can be micronized with a particle size volume distribution 50th percentile of 0.1-50 micron, or as larger macroscopic particles with particle size distribution volume 50th percentile of 0.1-3 mm, or a monolith (>3 mm) carbon substrate. In other embodiments, the carbon is porous with pore sizes <2 nm (microporous), 2-5 nm (mesoporous), or >5 nm (macroporous). In some embodiments, the dwell temperature to synthesize lithium carbide employing lithium is between 400-1000° C., or more preferably 600-900° C. In some embodiments, the dwell time to synthesize lithium carbide employing lithium hydride is between 0-12 hours or more preferably 0-2 hours. In some embodiments, the lithium hydride is in molar excess of the carbon thus resulting in formation of lithium-rich phases (e.g., Li₄C, Li₆C₂, LiC₃, Li₆C₃, Li₄C₃, Li₄C₅ and/or lithium metal in solid, liquid, or vapor phase). In other embodiments, the carbon is in molar excess of the lithium hydride thus resulting in carbon-rich domains of the resulting lithium carbide material.

### Example 12. Lithium Carbide Synthesis Employing Lithium Metal

In some embodiments, lithium carbide material is prepared by weighing out equimolar amounts of lithium metal and carbon powder in an inert environment consisting of either a noble gas (e.g., argon) or vacuum. The carbon can be micronized or be introduced as a larger macroscopic particle or monolith. The carbon surface can be modified using various reactive gas treatments: hydrogen, carbon dioxide, nitrogen, to enrich the surface composition in their respective elemental species. The powders are mixed until homogenous in color with a mortar and pestle and loaded into an inconel crucible. The material is then heated under an inert argon gas flow at a nominal flow rate (˜0.5 SLPM) in a horizontal tube furnace to a peak dwell temperature (900° C.) and allowed to dwell for a period of time (2 hours) for the reaction to complete, forming a pure lithium carbide phase. The crucible is then allowed to passively cool under inert atmosphere until room temperature is reached. The sample is then collected and micronized via attrition milling to a particle size below 25 um for characterization and electrochemical testing.

In some embodiments, the lithium metal takes the form of a metallic foil, an ingot, or a micronized powder. In some embodiments, the carbon structure is graphitic, nano-crystalline, or amorphous. In some embodiments, the carbon is porous with pore sizes <2 nm (microporous), 2-5 nm (mesoporous), or >5 nm (macroporous). In some embodiments, the carbon can be micronized with a particle size volume distribution 50th percentile of 0.1-50 micron, or be introduced as a larger macroscopic particle with particle size distribution volume 50th percentile of 0.1-3 mm or a monolith (>3 mm) carbon substrate. In some embodiments to synthesize lithium carbide from lithium metal, the dwell temperature is between 400-1000° C., or more preferably 700-900° C. In some embodiments to synthesize lithium carbide from lithium metal, the dwell time is between 0-12 hours or more preferably 0-2 hours. In some embodiments, the lithium metal is in molar excess of the carbon thus resulting in formation of lithium-rich phases (e.g., Li₄C, Li₆C₂, LisC3, Li₆C₃, Li₄C₃, Li₄C₅ and/or lithium metal in solid, liquid, or vapor phase). In other embodiments, the carbon is in molar excess of the lithium metal thus resulting in carbon-rich domains of the resulting lithium carbide material.

### Example 13. Lithium Carbide Indirect Route Synthesis Employing Lithium Containing Precursor that Converts to Lithium Hydride Intermediate

In some embodiments, the lithium carbide material is synthesized employing a lithium precursor that forms lithium hydride as an intermediate along the way to the lithium carbide material according to:

Li-R+C->LiH+C->(indirect route)

In such embodiments, lithium carbide compound powder can be prepared in an indirect manner in which a lithium containing precursor thermally decomposes into lithium hydride which then subsequently reacts with carbon to form lithium carbide compound. The reaction takes place in an inert atmosphere or under a reducing hydrogen atmosphere to promote the full decomposition of the precursor species to lithium hydride. The reaction then proceeds according to Example 1 above.

For such embodiments, example lithium containing precursors include, but are not limited to, lithium citrate, lithium acetate, n-butyllithium, lithium amide, lithium nitride, and phenyl lithium, and combinations thereof.

### Example 14. Carbon Pore Stabilized Lithium Carbide

In some embodiments, lithium carbide particles can be synthesized via carbon pores according to:

Li₂C₂+Carbon Scaffold->Pore stabilized carbide

In such embodiments, lithium carbide compound powder prepared from the above Examples can be melted and wetted into a porous carbon scaffold to improve a particular physical property (e.g., electrical conductivity) or performance attribute (e.g., electrochemical cycle stability). In a typical example lithium carbide compound material is mixed with a porous carbon scaffold and added into an inconel crucible then introduced into a horizontal tube furnace in an inert argon atmosphere. The sample is then heated to 500° C. and kept constant for a specified dwell time (1 hr) to allow for complete wetting of the scaffold and then subsequently cooled to room temperature and removed to a glovebox for analysis.

In some embodiments, the melt intrusion takes place in a stirred pressure vessel with a head space of argon. In some embodiments, the melt intrusion occurs at a temperature ranging from 400-900° C. for example 400-600° C. for example 450-550° C.

### Example 15. Lithium Carbide Synthesis from Organolithium Solutions

In some embodiments, lithium carbide particles can be synthesized by contacting an alkyne functionalized hydrocarbon with a Li-arene solution. Without being bound by theory, such synthesis allows to forgo particle size diminution.

In some embodiments, lithium carbide material can be prepared via a liquid-gas contact reaction as follows. In a typical embodiment, a 1 molar solution of lithium biphenyl is prepared by dissolving lithium metal in a solution of 1 molar polycyclic aromatic hydrocarbon (biphenyl) in an ether (tetrahydrofuran) solvent under an inert gas (argon) atmosphere. Acetylene gas is then introduced to the solution wherein a spontaneous chemical reaction takes place precipitating lithium carbide compound powder and evolving hydrogen gas as a byproduct. The lithium carbide compound is subsequently washed and collected from the solution through rinsing and centrifugation with pure ether. The sample is then vacuum dried to yield the free-flowing lithium carbide compound powder.

In some embodiments, lithium carbide compounds can be prepared via a coprecipitation reaction as follows. A solution containing an alkyne functionalized hydrocarbon is slowly added to the lithium-arene solution wherein upon contact a spontaneous reaction occurs forming the lithium carbide compound and evolving hydrogen gas as a byproduct. The lithium carbide compound is subsequently washed and collected from the solution through rinsing and centrifugation with pure ether. The sample is then vacuum dried to yield the free-flowing lithium carbide compound powder.

In some embodiments, the concentration of lithium in the solution is between 0.01-4 mol/L, for example 0.1-1 mol/L. In some embodiments, the reaction mixture comprises a polycyclic aromatic hydrocarbon, and examples include, but are not limited to, naphthalene, methyl naphthalene, methyl biphenyl, dimethyl biphenyl, trimethyl biphenyl, tetramethyl biphenyl, terphenyl, and pyrene, and combinations thereof. In some embodiments, the reaction mixture comprises an ether solvent, and examples include, but are not limit to, methyl-tetrahydrofuran, diethyl ether, dioxolane, dioxane, dimethoxyethane, diglyme, triglyme, and tetraglyme, and combinations thereof.

### Example 16. Lithium Carbide Synthesis from the Solid State in a Moving Bed

In some embodiments, the lithium carbide compound material can be prepared in a reaction vessel wherein the reactant materials are kept in constant motion to minimize adhesion to the materials of construction (e.g., reactor walls, crucibles, etc.). Such reactor configurations may include fluidized bed reactors wherein the carbon and lithium hydride reactants are kept in motion using gas flow fluidization allowing them to react and form discrete lithium carbide compound particles. In yet another embodiment the reactor configuration is a rotating horizontal tube furnace with or without the presence of a milling media to keep the reactant powders cascading as they react to form discrete lithium carbide compound particles.

### Example 17. Lithium Carbide Synthesis from Carbon Dioxide and Lithium Metal

In another embodiment, the lithium carbide material can be prepared through a gas-solid interaction wherein lithium metal is heated in the presence of carbon dioxide according to the reaction scheme:

10Li+2CO₂->Li₂C₂₊₄Li₂O

In a typical synthesis, lithium metal is heated above its melting point (>180° C.) in a closed vessel. Subsequently carbon dioxide either already present in the atmosphere of the vessel or externally flowed into the vessel in molar excess reacts with the molten lithium metal spontaneously yielding a mixture of lithium carbide compound and lithium oxide. The lithium carbide compound can then be collected and purified using techniques known in the art.

### Example 18. Lithium Carbide Synthesis from Hydrocarbon Gas and Lithium Metal

In some embodiments, the lithium carbide compound material can be prepared through a gas-solid interaction wherein lithium metal is heated in the presence of a hydrocarbon gas as the carbon precursor. Example hydrocarbon gases in this regard include alkanes, alkenes, and alkyne hydrocarbon gasses. In a preferred embodiment, the gas is ethylene, and the reaction proceeds according to the reaction scheme:

6Li+C₂H₄->Li₂C₂+4LiH

In a typical synthesis, lithium metal is heated above its melting point (>180° C.) in a closed vessel. Subsequently ethylene either already present in the atmosphere of the vessel or externally flowed into the vessel in molar excess reacts with the molten lithium metal spontaneously yielding a mixture of lithium carbide compound and lithium hydride. The lithium carbide compound can then be collected and purified using techniques known in the art.

In some embodiments, a second carbon precursor (besides the hydrocarbon gas) is also present, for example porous carbon material, such as porous carbon particles. In such embodiments, the second carbon precursor is also in contact with the lithium metal so as to react with lithium hydride and also produce lithium carbide material, thereby further improving overall yield.

### Example 19. Heteroatom Doped Lithium Carbide Material

In some embodiments, a heteroatom dopant can be introduced to the lithium carbide material in any one of the synthesis procedures described in the above Examples. The presence of the heteroatom dopant can act to balance the oxidation state and stabilize the carbide structure as lithium is extracted/inserted during charge/discharge cycling in a Li-ion battery. In one example synthesis procedure the heteroatom dopant is sodium. Wherein a 1:1 molar ratio of lithium metal and sodium metal is co-dissolved in an equimolar solution of biphenyl dissolved in tetrahydrofuran solvent. Acetylene gas is subsequently bubbled through this solution in molar excess to spontaneously form a lithium sodium carbide composite with the stoichiometry Li₍₁₋ₓ₎NaₓC₂. The lithium sodium carbide composite is then collected from the solution according to the above Examples for analysis and electrode fabrication.

In some embodiments, the heteroatom dopant is an alternative alkali metal such as potassium, rubidium, or cesium. In some embodiments, the heteroatom dopant comprises an alkaline metal such as beryllium, magnesium, calcium, strontium, or barium. In some embodiments, the heteroatom dopant comprises a transition metal such as titanium, niobium, tantalum, tungsten, molybdenum, yttrium, chromium, iron, cobalt, nickel, copper, zinc, cadmium, mercury, indium, gallium, aluminum, germanium, tin, lead, bismuth, antimony, thallium, or selenium. In some embodiments, the heteroatom dopant comprises a noble metal such as ruthenium, osmium, rhodium, iridium, palladium, platinum, silver, or gold. In some embodiments, the heteroatom dopant comprises a non-metal such as silicon, boron, nitrogen, oxygen, sulfur, phosphorus, arsenic, or hydrogen.

In some embodiments, the stoichiometry of the heteroatom doped lithium carbide compound is represented as Li₍₁₋ₓ₎MₓC₂ where M=heteroatom dopant and x is between 0-1.

TABLE 4
Calculated gravimetric capacities of example derivatives and
heteroatom stoichiometries of lithium carbide materials.
Heteroatom doped lithium carbide materials
and gravimetric capacity in mAh/g

| Li₂MnC₄ | LiCaC₃ | Li₂CaC₄ | LiKC₂ | LiNaC₂ | Li₂CN₂ | Li₃BN₂ | Li₂Al₄C₅ | Li₂Ti₃C₂ |
|---|---|---|---|---|---|---|---|---|
| 459 | 323 | 688 | 383 | 497 | 995 | 1348 | 346 | 295 |

Reference materials and gravimetric capacity in mAh/g

| Li_{3.75}Si | LiC₆ | LiFePO₄ | Li₂C₂ |
|---|---|---|---|
| 3579 | 372 | 169.9 | 1415 |

In some embodiments, the heteroatom doped lithium carbide compound can have two heteroatom dopants. For the example of divalent heteroatom doping, the according stoichiometry is Li_{(1-x-y)}MₓZ_{y}C₂ where M=heteroatom dopant 1 and Z=heteroatom dopant 2.

In some embodiments, the heteroatom doped lithium carbide compound can have three heteroatom dopants. For the example of trivalent heteroatom doping, the according stoichiometry is Li_{(1-x-y-z)}MₓZ_{y}Y_{z}C₂ where M heteroatom dopant 1 and Z heteroatom dopant 2, and Y=heteroatom dopant 3.

Additional embodiments are envisioned, for example tetravalent heteroatom doped lithium carbon materials.

### Example 20. Production of Various Lithium Carbide Materials

A variety of lithium carbide compounds were synthesized as described in Table 5 following routes as described in Example 11 and Example 12. For Samples LCC4 and LCC19 the lithium precursor was lithium metal, and for all other Samples in Table 5, the lithium precursor was LiH. For Sample LCC22, the form factor was a monolith, and for all other Samples in Table 5, the form factor was powder. Typical reaction time to form the lithium carbide compound was 2 h. For Samples produced with Ar, the furnace pressure was ambient. For Samples produced with vacuum, the furnace pressure was sub ambient, typically in the range of 0.001 to 100 Torr. In some embodiments, the pressure can vary from 0.01 to 10 Torr, for example 0.01 to 1 Torr. (Add text around orange color phenomenon)

TABLE 5
Examples of synthesized lithium carbide compounds.

| | | | | | Melting | Enthalpy |
|---|---|---|---|---|---|---|
| | | Li:C | Reaction | | point by | of |
| Example | Carbon | ratio | Temp | Furnace | DSC | fusion |
| Name | Scaffold | mol:mol | (° C.) | Atmosphere | (° C.) | (J/g) |
| LCC1 | Carbon 1 | 0.4:1 | 900 | Vacuum | Pending | Pending |
| LCC2 | Carbon 1 | 0.4:1 | 900 | Vacuum | Pending | Pending |
| LCC3 | Carbon 1 | 0.4:1 | 900 | Vacuum | Pending | Pending |
| LCC4 | Carbon 1 | 0.25:1 | 440 | Vacuum | Pending | Pending |
| LCC5 | Carbon 1 | 0.5:1 | 900 | Vacuum | Pending | Pending |
| LCC6 | Carbon 1 | 1:1 | 900 | Argon | Pending | Pending |
| LCC7 | Carbon 1 | 1:1 | 900 | Argon | Pending | Pending |
| LCC8 | Carbon 1 | 1:1 | 900 | Argon | 447.88 | 179.07 |
| LCC9 | Carbon 1 | 1:1 | 700 | Argon | Pending | Pending |
| LCC10 | Carbon 2 | 1:1 | 900 | Argon | 449.1 | 183.41 |
| LCC11 | Carbon 2 | 1:1 | 700 | Argon | Pending | Pending |
| LCC12 | Carbon 3 | 1:1 | 900 | Argon | 451.16 | 191.84 |
| LCC13 | Carbon 3 | 1:1 | 700 | Argon | Pending | Pending |
| LCC14 | Carbon 4 | 1:1 | 900 | Argon | 460.02 | 185.55 |
| LCC15 | Carbon 4 | 1:1 | 700 | Argon | Pending | Pending |
| LCC1 | Carbon 1 | 0.4:1 | 900 | Vacuum | Pending | Pending |
| LCC2 | Carbon 1 | 0.4:1 | 900 | Vacuum | Pending | Pending |
| LCC3 | Carbon 1 | 0.4:1 | 900 | Vacuum | Pending | Pending |
| LCC16 | Carbon 5 | 1:1 | 900 | Argon | 450.54 | 149.16 |
| LCC17 | Carbon 5 | 1:1 | 900 | Argon | Pending | Pending |
| LCC18 | Carbon 5 | 1:1 | 700 | Argon | Pending | Pending |
| LCC19 | Carbon 1 | 1:1 | 900 | Argon | 445.80 | 208.09 |
| LCC20 | Carbon 6 | 1:1 | 900 | Argon | 453.38 | 145.86 |
| LCC21 | Carbon 7 | 1:1 | 900 | Argon | 455.01 | 174.67 |
| LCC22 | Carbon 8 | 1:1 | 900 | Argon | Pending | Pending |

### Example 21. Production of Various Lithium Carbide Materials Employing Heteroatom Doping

A variety of lithium carbide compounds were synthesized as described in Table 6 following routes as described in Example 19.

TABLE 6
Examples of heteroatom doped lithium carbide compounds.

| | | | | | Reac- |
|---|---|---|---|---|---|
| | | | | Reac- | tion |
| | | | | tion | dwell |
| Example | Secondary | Carbon | Li:Metal:Carbon | Temp | time |
| Name | Metal | Scaffold | mol:mol:mol | (° C.) | (hr) |
| LCC23 | Aluminum | Carbon 1 | 1:1:1 | 900 | 2 |
| LCC24 | Aluminum | Carbon 1 | 1:1:1 | 900 | 2 |
| LCC25 | Aluminum | Carbon 1 | 1:1:4 | 900 | 2 |
| LCC26 | Sodium | Carbon 1 | 1:1:2 | 600 | 2 |
| LCC27 | Sodium | Carbon 1 | 1.9:0.1:2 | 700 | 2 |
| LCC28 | Sodium | Carbon 1 | 1.9:0.1:2 | 700 | 3 |
| LCC29 | Calcium | Carbon 1 | 2:1:4 | 900 | 2 |
| LCC30 | Sodium | Carbon 1 | 1:1:2 | 700 | 0.5 |
| LCC31 | Calcium | Carbon 1 | 3.9:0.1:4 | 900 | 2 |
| LCC32 | Calcium | Carbon 1 | 1:1:3 | 900 | 2 |
| LCC33 | Manganese | Carbon 1 | 2:1:4 | 900 | 8 |
| LCC34 | Manganese | Carbon 1 | 3.9:0.1:4 | 900 | 2 |

### Example 22. Production of Various Lithium Carbide Materials Via Synthesis at Gas-Liquid Interface

A variety of lithium carbide compounds were synthesized as described in Table 7 following routes as described in Example 15. For Samples LCC35 and LCC36 the total metal ion concentration in solution was 2M. For all other Samples the total metal ion concentration was 1.5M. Sample LCC40 was synthesized with a secondary alkali metal at 1:1 molar ratio to lithium. Samples were typically heat treated at 350° C. after drying of collected powder.

TABLE 7
Examples of lithium carbide compounds
synthesized at a gas-liquid interface.

| | | | Acetylene | Reac- | |
|---|---|---|---|---|---|
| | | | flow | tion | Wash- |
| Example | Carrier | | rate | time | ing |
| Name | Solvent | Arene | (SCCM) | (min) | solvent |
| LCC35 | THF | 1-methylnaphthalene | 50 | 120 | THF |
| LCC36 | THF | 1-methylnaphthalene | 50 | 90 | THF |
| LCC37 | THF | 1-methylnaphthalene | 75 | 50 | THF |
| LCC38 | DME | 1-methylnaphthalene | 50 | 15 | THF |
| LCC39 | THF | 1-methylnaphthalene | 100 | 140 | THF |
| LCC40 | THF | 1-methylnaphthalene | 100 | 135 | THF |
| LCC41 | DME | 1-methylnaphthalene | 125 | 200 | DME |

### Example 23. Gas Sorption Analysis Accomplished without Exposing Sample to Air

Gas sorption analysis is a standard analysis technique for studying material porosity and surface area. To this end for air-sensitive (oxygen-sensitive) materials such as the lithium carbide materials., samples are collected in an argon glove box and sealed in a frit capped sample tube. The sample is measured using a Micromeritics Tristar II+ instrument using nitrogen adsorption method.

### Example 24. Differential Scanning Calorimetry of Lithium Carbide Materials

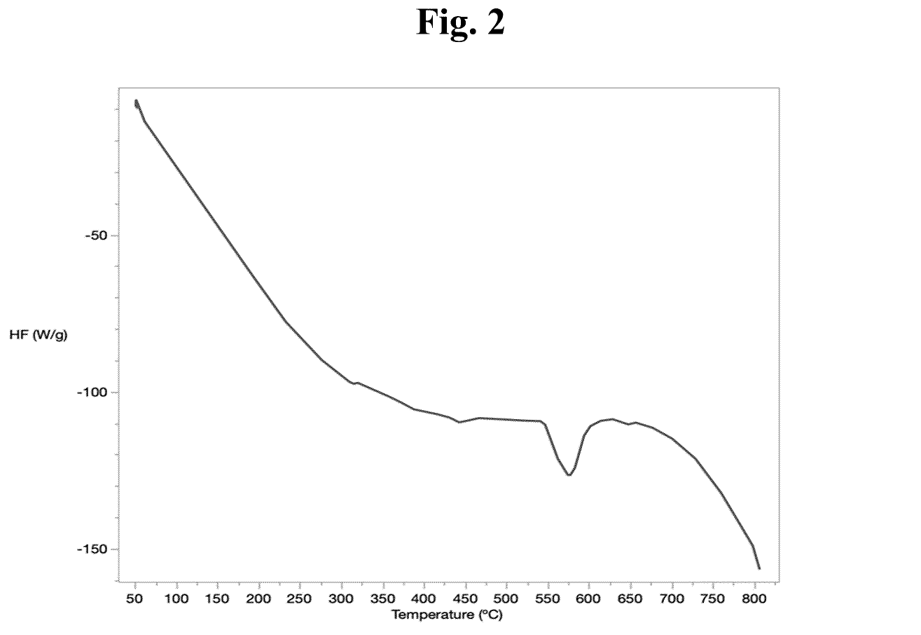

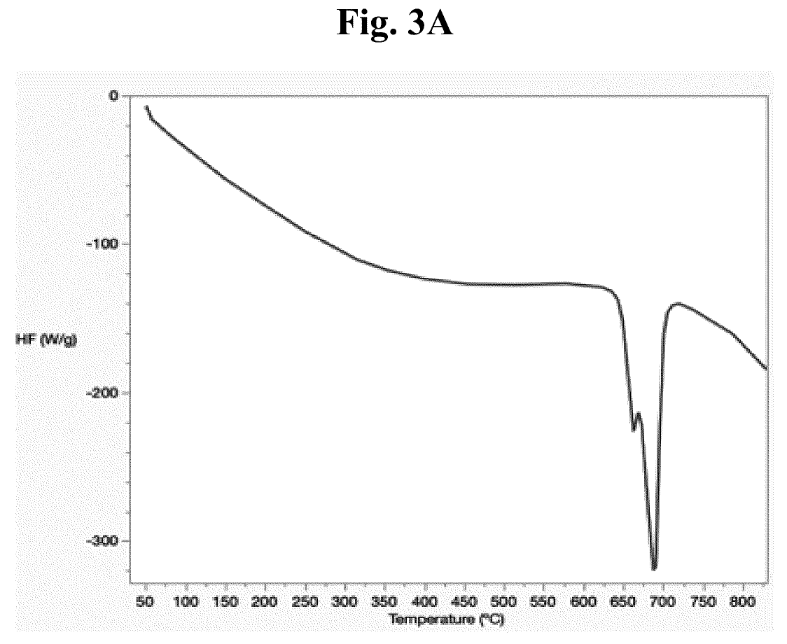

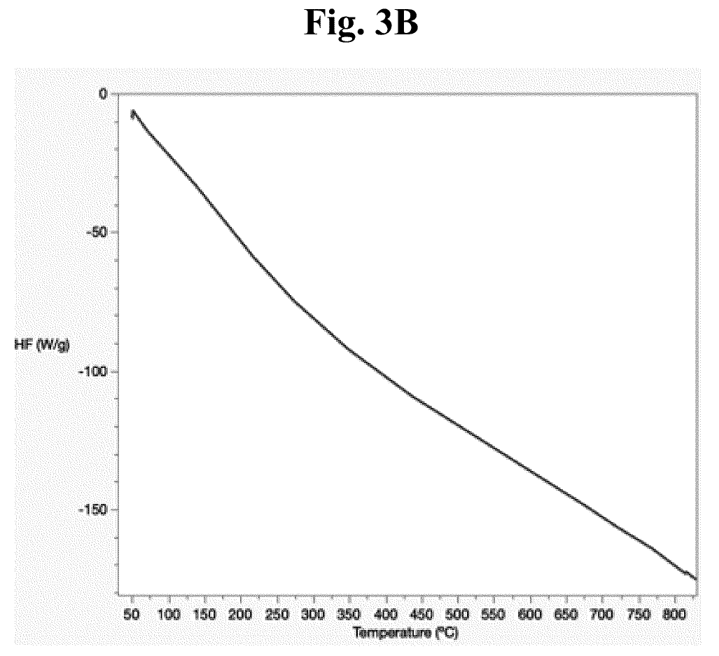

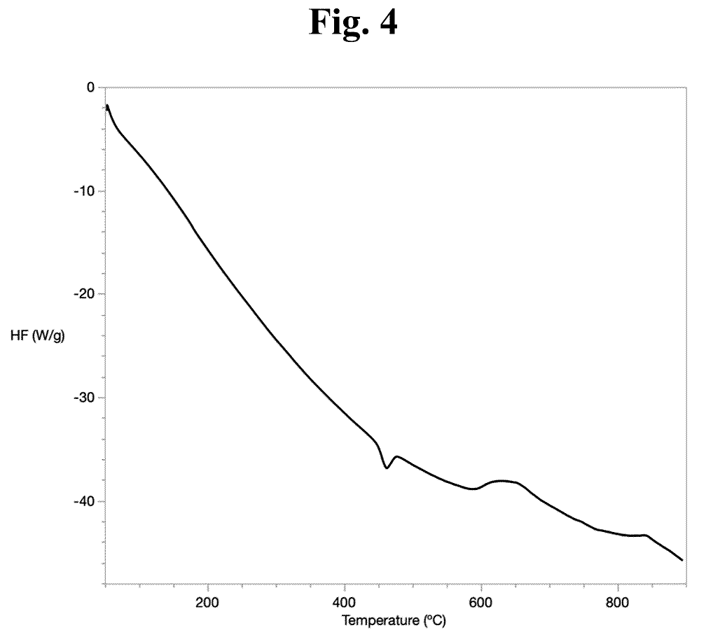

Differential scanning calorimetry is used for determination of the melting temperature and reaction enthalpy of lithium carbide compound. In a typical analysis, a small portion (˜1-50 mg) of the lithium carbide compound prepared according to Examples 1-10 is loaded into stainless steel crucibles and sealed in an argon atmosphere. The crucibles are analyzed on a Mettler Toledo TGA/DSC 3+ instrument using an empty stainless steel crucible as the reference. The crucible is heated to 900° C. at a heating rate of 10° C./min to yield a heat flow versus temperature curve wherein endothermic events (e.g., melting) appear as negative “valleys” in the curve while exothermic events appear as positive “peaks”. FIG. 2 shows an example heat flow curve detailing the endothermic reaction (at ˜550° C.) between lithium hydride and carbon powder according to Example 13 to form lithium carbide compound. Heat flow curves of lithium hydride and carbon powder are shown in FIGS. 3A and 3B. This shows the characteristic melting event of lithium hydride (˜690° C.) and no reactions or phase changes in the case of the carbon powder, respectively. FIG. 4 shows a typical characteristic melting point of an exemplary lithium carbide compound material (LCC8) at ˜450 C.

### Example 25. X-Ray Diffraction (XRD) of Lithium Carbide Materials for Crystalline Phase Determination

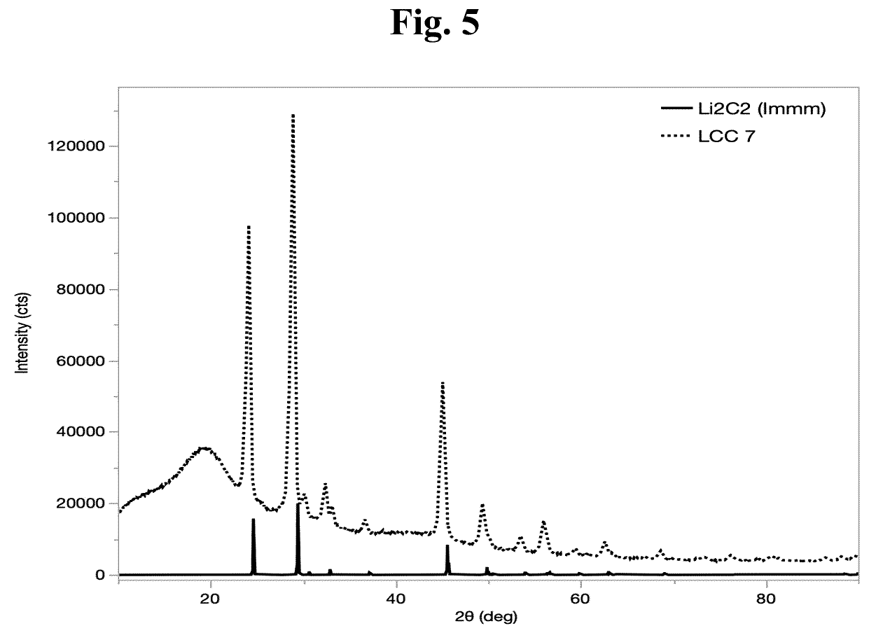

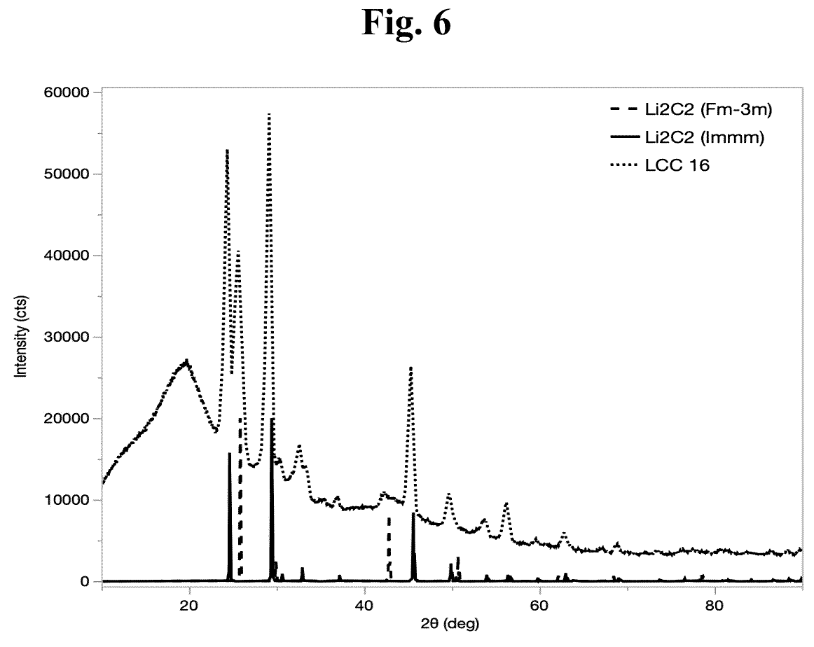

X-ray diffraction (XRD) is used for determination of the crystalline structure of lithium carbide compounds. In a typical analysis a sample is collected and sealed in an X-Ray transparent sample vessel in an argon atmosphere. The sample is then measured using a Bruker D8 Advance instrument with a 1600 W copper excitation source and 0.5 mm primary slit. The sample is measured by a continuous coupled theta/2theta scan across a 2-theta range of 10-90° at a 2-theta rate of 0.042 degrees per second. Peaks were then matched with NIST database records for crystalline species known in the art. FIG. 5 shows a diffraction pattern of an exemplary lithium carbide material (LCC-7). It is clear that the material forms a crystalline lithium carbide phase with a lmmm space group. In some instances, an additional lithium carbide space group (Fm-3m) is present as seen for another exemplary lithium carbide material (LCC-8) in FIG. 6.

### Example 26. Colorimetry of Lithium Carbide Materials for Crystalline Phase Determination

The visible color of lithium carbide materials according to Example 12 was measured using a spectrophotometer with the convention known in the art as the CIELab 3-dimensional color space under industry standard D65 illumination. Wherein, the L-value represents the lightness (100=white, 0=black), the a-value represents red to green (+=redder, −=more green), and b-value represents yellow to blue (+=more yellow, −=more blue).

### Example 27. Electrochemical Characterization of Lithium Carbide Materials in Half Cell Coin Cells

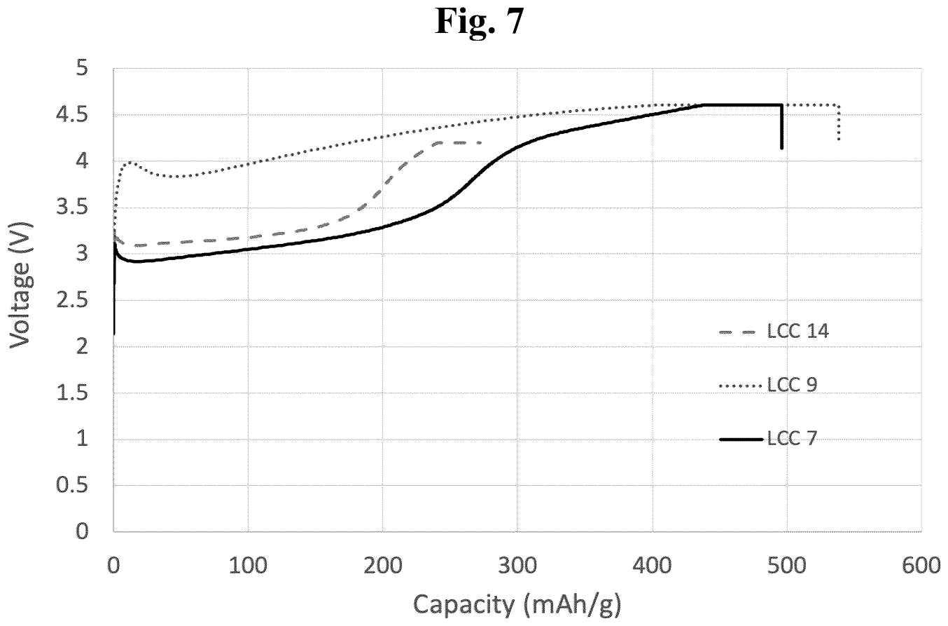

Lithium carbide compound material is casted into an electrode sheet using the following recipe: 60% to 95% active material,20% to 1% conductive enhancer,20% to %4 binder, wherein the active material can be lithium carbide, or a mix of lithium carbide with conventional cathode materials such as NCA, NCM811, LFP, LCO, etc; Binder can be either polyvinylidene fluoride (PVDF), polyacrylonitrile (PAN), polytetrafluoroethylene (PTFE), or a combination of them in a non-aqueous solvent such as dimethyl formamide (DMF), dimethylsulfoxide (DMSO), N-methyl-2-pyrrolidone (NMP), etc; Conductive enhancer can be carbon black, hard carbon, carbon nanotubes, etc. Electrochemical performance of lithium carbide material is measured in a half-cell coin cell configuration with lithium metal as the reference and counter electrode. LiPF₆ salt dissolved in carbonate based solvents is used as the electrolyte. Typical polymer separator such as PP membrane or a trilayer PP/PE/PP is used in the cell. Cells are tested in a battery cycler using a constant current charge/discharge test protocol. Cells are charged (extraction of lithium) at C/10 rate to an upper cut-off voltage of 4.2V or 4.6V, held at the upper cut-off voltage (constant voltage) until the current drops to C/20 rate and then discharged (insertion of lithium) at C/10 rate to a cut-off voltage of 2.5V or 2.0V. FIG. 7 depicts the first cycle extraction capacity for three exemplary lithium carbide materials (LCC-7, LCC-9, LCC-14).

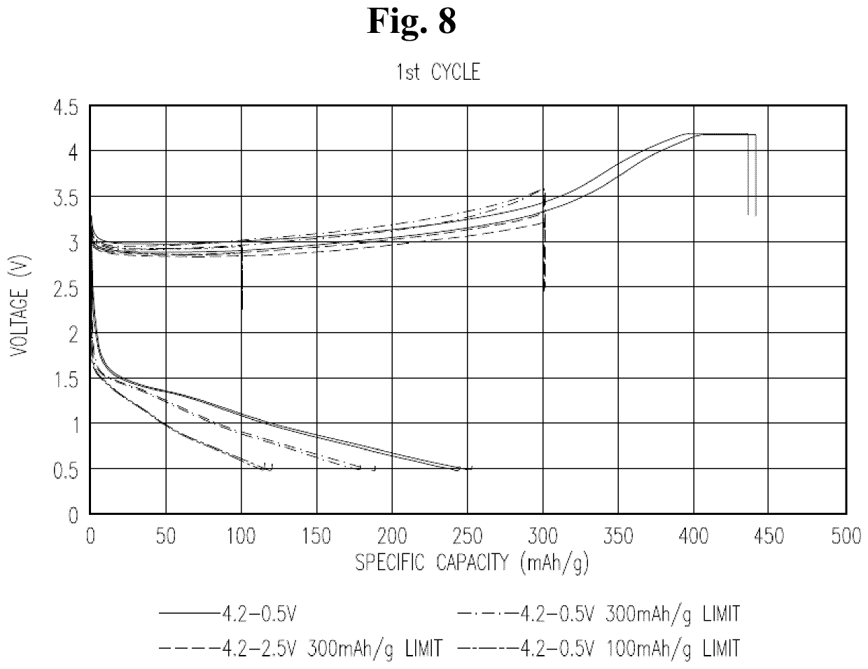

Cycling of batteries comprising the lithium carbide compound material can also be voltage limited. To this end, in a half-cell the lower value for the voltage window can be varied, for example 2.5V, or 2.0V, or 1.8V, or 1.6V, or 1.4V, or 1.2V, or 1.0V, or 0.8V, or 0.6V, or 0.5V. This lower limit in a half cell can be translated to a corresponding voltage in a full cell battery as known in the art. FIG. 8 depicts the voltage profile of lithium carbide sample LCC16 showing exaction capacity from 4,2V and insertion capacity down to 0.5V (vs. Li/Li+) at different capacity limitations.

FIG. 8 depicts the voltage profile of lithium carbide Sample LCC 16 showing extraction capacity up to 4.2V and insertion capacity down to 0.5V (vs. Li/Li+) at different capacity limitations. FIG. 8 demonstrates significant loss in insertion capacity indicating irreversible changes during initial cycling despite limiting extraction capacity to minimize degradation of the lithium carbide. Compared to FIG. 7, Li-ion insertion behavior was only observed at <2V vs. Li/Li+ with a large (˜2V) hysteresis. At these low voltages, contributions from other anode active components (e.g., carbon black, SEI) in the system can result as evidenced in the 100 mAh/g capacity limited curve wherein more lithium was inserted than initially extracted.

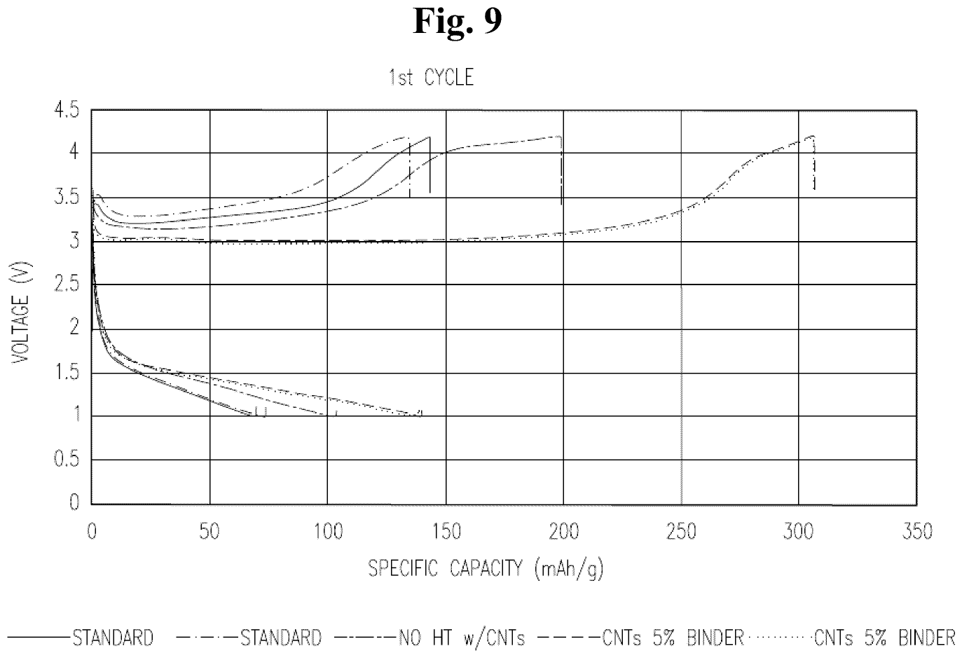

FIG. 9 demonstrates how the addition of carbon nanotubes (CNT) can improve electrochemical performance of the lithium carbide compound. In particular, the addition of 5 wt % CNT results in an extraction capacity increase by ˜2× and extraction voltage reduced by ˜0.5V thereby increasing both energy density and conductivity. The CNT's act to increase electrical conductivity between particles thereby improving kinetics associated with electrochemical lithiation.

### Example 28. Employment of the Lithium Carbide Compound as a Cathode Additive for Pre-Lithiation of Anode

Owing to its high capacity and operating voltage, the lithium carbide compound can be blended with commonly employed Li-ion cathode materials (e.g., LiFePO₄, LiCoO₂, LiNi_{0.8}Mn_{0.1}Co_{0.1}O₂, LiNi_{0.8}Co

Owing to its high capacity and operating voltage, the lithium carbide compound can be blended with commonly employed Li-ion cathode materials (e.g., LiFePO₄, LiCoO₂, LiNi_{0.8}Mn_{0.1}Co_{0.1}O₂, LiNi_{0.8}Co_{0.15}Al_{0.05}O₂) during the slurry and electrode preparation process to act as a sacrificial Li-ion donor to compensate for capacity losses in the first charge/discharge cycle. An example cell-level energy density improvement is demonstrated in Table 8 below.

TABLE 8
Example Li-ion battery active material composition both with (“w/prelith”) and
without (“w/o prelith”) a lithium carbide compound additive to improve cell energy density.

| | | | Active | Active | | | | |
|---|---|---|---|---|---|---|---|---|
| | | Active | Material | Material | Cell | Cell | | Cell |
| | | Material | Reversible | 1st cycle | charge | discharge | 1st | Specific |
| | Active | Mass | Capacity | capacity | capacity | capacity | Cycle | Capacity |
| | Material | (g) | (mAh/g) | (mAh/g) | (mAh) | (mAh) | Efficiency | (mAh/g) |
| w/o | Cathode | 9 | 200 | 215 | 1935 | 1260 | 93% | 126 |
| prelith | Anode | 1 | 1400 | 2000 | 2000 | | 70% | |
| w/ | Cathode | 6.51 | 200 | 215 | 1400 | 1302 | 93% | 149 |
| prelith | Anode | 1 | 1400 | 1400 | 1400 | | 100% | |
| | LCC | 1.20 | | 500 | 600 | | N/A | |

In the example shown in Table 8, an anode is chosen with high specific capacity (e.g., silicon) and a cathode material (e.g., NMC811) is paired against that anode in a 10-fold mass excess in order to balance the capacity of the cell which has an absolute capacity of 2000 mAh. Upon the first charge/discharge cycle an intrinsic loss in capacity is observed in the cell caused by SEI/CEI formation and other parasitic losses corresponding to 93% and 70% for the cathode and anode, respectively. These losses are compounded resulting in a total cell first cycle efficiency of 65%. The discharge (i.e., reversible) capacity of the cell now being 1260 mAh divided by the mass of combined active materials results in a cell-level capacity of 126 mAh/g. To compensate for that loss, the lithium carbide compound with a specific charge capacity of 500 mAh/g is blended with the cathode. As a result, the lithium carbide compound contributes all of its capacity on the cell's first charge cycle resulting in a 18% cell specific capacity improvement even when taking into account the added mass of the lithium carbide compound material.

The lithium carbide compound has several key advantages over state of the art cathode prelithiation additives: (i) it offers a very high theoretical capacity of 1415 mAh/g compared to Li₂O₂ (1168 mAh/g) and LisFeO₄ (867 mAh/g); (ii) the electrochemical decomposition byproduct is a solid (amorphous carbon) compared to gaseous byproducts such as O₂, H₂, and N₂ (for Li₂O₂, LiH, and Li₃N, respectively) which can cause cell swelling if not degassed properly; (iii) the amorphous carbon byproduct it does leave behind can contribute to conductivity improvements akin to a carbon black additive commonly employed in Li-ion battery electrodes as conductivity enhancers, and; (iv) the operating voltage for extraction of the lithium-ions is quite low (<3.5V vs. Li/Li+) as opposed to LiH and Li₃N which require >4.4V vs. Li/Li+ to extract capacity that can lead to oxidation of the electrolyte at such high voltage.

### Example 29. Employment of the Lithium Carbide Compound as a Reducing Agent

A lithium carbide compound as described above is useful as a reducing agent in various chemical reactions. Lithium carbide compounds, such as those described above, function in a similar manner to other organolithium reagents in which the negative redox potential of the ground state lithium present in the molecule reacts with a wide variety of candidates. For instance, in one embodiment, the lithium carbide compound reacts in a reduction reaction to reduce metal halides (MX) to the elemental metal (M) and the corresponding lithium halide (LiX). In yet another embodiment, the lithium carbide compound may reduce nitrogen based functional groups of a compound to an amine functional group. An example reduction reaction is shown below:

Li₂C₂+MX→M+LiX+C

### Example 30. Employment of the Lithium Carbide Compound for Acetylene Storage

Lithium carbide compounds described above may serve as a source for acetylene gas on demand. In this example, a lithium carbide is reacted with water to produce acetylene and lithium oxide. In this example, acetylene may be stored via the lithium carbide compound in a molar density of 34.2 mol/L rather than the acetylene gas byproduct of the reaction which has a molar density of 0.04 mol/L at standard temperature and pressure (STP).

Li₂C₂+H₂O=C₂H₂+Li₂O

Further, the lithium carbide particle size and/or surface area is optimized for a desired acetylene generation rate.

### Example 31. Employment of the Lithium Carbide Compound in the Process of Radiocarbon Dating

In this example, lithium carbide compounds are created by the melt infusion method of synthesis for lithium carbide compounds (LCC) (ex. 2), the vapor phase method of synthesis for lithium carbide compounds (ex. 4), the reduction of lithium salts methods for synthesis of lithium carbide compounds (ex. 6), or the electrochemical methods of forming lithium carbide compounds (ex. 7). The lithium carbide compound(s) is then reacted with water to produce acetylene. The isotope mass of the acetylene produced is then correlated to the age of the initial carbon material. The rate of acetylene produced is measured by mass spectroscopy which will separate the signal produced into distinct peaks with corresponding C₁₄ atoms present in the original carbon material. Ultimately by comparing the ratio of peak intensities a carbon ratio can be determined for a sample which is used to determine age.

### EMBODIMENTS

Embodiment 1. A lithium carbide material comprising a stoichiometry of LiₓC_{y}M_{z} wherein x ranges from greater than 0 to 8, y ranges from greater than 0 to 5, M is an element selected from transition metals, metalloids, Group 1, Group 2, Group13, Group15, and Group 17, and z ranges from 0 to 8.

Embodiment 2. A lithium carbide material comprising a stoichiometry of LiₓC_{y}M_{z} wherein x ranges from greater than 0 to 2, y ranges from greater than 0 to 2, M is an element selected from transition metals, metalloids, Group 1, Group 2, Group13, Group15, and Group 17, and z ranges from 0 to 2.

Embodiment 3. A lithium carbide material comprising a stoichiometry of LixCy wherein x is 2 and y is 2.

Embodiment 4. A lithium carbide material comprising a stoichiometry of LiₓC_{y}M_{z} wherein x ranges from greater than 0 to 2, y is 2, M is an element selected from transition metals, metalloids, Group 1, Group 2, Group13, Group15, and Group 17, and z ranges from 0 to 2.

Embodiment 5. A composite comprising any of the lithium carbide materials according to any of Embodiments 1 through 4 and also comprising an additional lithium compound selected from lithium metal, lithium salts selected from lithium oxides, lithium nitrides, lithium halides, organolithium compounds, and lithium alloys, and combinations thereof.

Embodiment 6. A composite comprising any of the lithium carbide materials according to any of Embodiments 1 through 4 and also comprising an additional carbon compound selected from amorphous carbon, graphite, graphene, soft carbon, and hard carbon, and combinations thereof.

Embodiment 7. A composite comprising any of the lithium carbide materials according to any of Embodiments 1 through 4 and also comprising an addition species selected from carbon, lithium, lithium compounds selected from lithium salts selected from lithium oxides, lithium nitrides, lithium halides, organolithium compounds, and lithium alloys, and an additional carbon compound selected from amorphous carbon, graphite, graphene, soft carbon, and hard carbon, and combinations thereof.

Embodiment 8. A composite according to Embodiment 6 wherein the additional carbon compound is a carbon coating of the surface of the lithium carbide compound.

Embodiment 9. A composite according to Embodiment 5 wherein the additional lithium compound is lithium metal.

Embodiment 10. A lithium carbide compound according to any of Embodiments 1 through 4 wherein the lithium carbide compound comprises a surface coating comprising a metal oxide selected from Al₂O₃, TiO₂, ZrO₂, Li₂O, ZnO, SiO₂, and combinations thereof.

Embodiment 11. A lithium carbide compound according to any of Embodiments 1 through 4 wherein the lithium carbide compound comprises a surface coating comprising a halide salt.

Embodiment 12. A lithium carbide compound according to any of Embodiments 1 through 4 wherein the lithium carbide compound comprises a surface coating comprising lithium fluoride.

Embodiment 13. A lithium carbide compound according to any of Embodiments 1 through 4 wherein the lithium carbide compound comprises a surface passivation layer comprising end groups comprising oxygen, hydrogen, nitrogen, halogens, and combinations thereof.

Embodiment 14. A lithium carbide compound according to any of Embodiments 1 through 4 wherein the lithium carbide compound comprises a surface passivation layer comprising end groups comprising fluorine.

Embodiment 15. An electrode comprising a lithium carbide compound according to any of Embodiments 1 through 14.

Embodiment 16. A lithium carbide battery comprising a cathode comprising a lithium carbide compound according to any of Embodiments 1 through 14.

Embodiment 17. A lithium carbide battery comprising a cathode comprising a lithium carbide compound according to any of Embodiments 1 through 14, and comprising an anode comprising a silicon carbon composite material.

Embodiment 18. A method to prelithiate an anode in operando comprising irreversible extraction of lithium from a cathode comprising a lithium carbide compound according to any of Embodiments 1 through 14 to an anode. An operando battery process refers to a process that occurs in the regular operation of the battery. Such as for instance the prelithiation of an anode from the cathode during the first cycle of battery operation.

Embodiment 19. The lithium carbide compound according to any of Embodiments 1 through 14 wherein the compound is a particulate material comprising a Dv50 between 0.1 to 50 microns.

Embodiment 18. The lithium carbide compound according to Embodiment 19 wherein the particle shape is spheroidal.

Embodiment 19. The lithium carbide compound according to Embodiment 8 wherein the carbon coating is an amorphous carbon layer achieved by chemical vapor deposition of a hydrocarbon gas.

Embodiment 20. The lithium carbide compound according to any of Embodiments 1 through 14 wherein the compound comprises a capacity of greater than 150 mAh/g.

Embodiment 21. The lithium carbide compound according to any of Embodiments 1 through 14 wherein the compound comprises a capacity of greater than 200 mAh/g.

Embodiment 22. The lithium carbide compound according to any of Embodiments 1 through 14 wherein the compound comprises a capacity of greater than 300 mAh/g.

Embodiment 23. The lithium carbide compound according to any of Embodiments 1 through 14 wherein the compound comprises a capacity of greater than 500 mAh/g.

Embodiment 24. The lithium carbide compound according to any of Embodiments 1 through 14 wherein the compound comprises a capacity of greater than 800 mAh/g.

Embodiment 25. The lithium carbide compound according to any of Embodiments 1 through 14 wherein the compound comprises a capacity of greater than 1000 mAh/g.

Embodiment 26. The lithium carbide compound according to any of Embodiments 1 through 14 wherein the compound comprises a capacity of greater than 1200 mAh/g.

Embodiment 27. The lithium carbide compound according to any of Embodiments 1 through 14 wherein the compound comprises a capacity of greater than 1400 mAh/g.

Embodiment 28. A method of manufacturing a lithium carbide compound, the method comprising:
a. mixing a solid carbon precursor and a solid lithium precursor;
b. heating the mixture at 180° C. to 1300° C. under an inert environment to melt the lithium precursor and react to form the lithium carbide compound;
c. cooling under an inert environment.

Embodiment 29. A method of manufacturing a lithium carbide compound according to Embodiment 28 further comprising particle size reduction of the lithium carbide compound under an inert environment.

Embodiment 30. A method of manufacturing a lithium carbide compound according to Embodiment 28 or Embodiment 29 wherein the inert environment is a noble gas.

Embodiment 31. A method of manufacturing a lithium carbide compound according to Embodiment 28 or Embodiment 29 wherein the inert environment is a vacuum.

Embodiment 32. A method of any of Embodiments 28 through 31 further comprising mixing a third precursor comprising an element selected from transition metals, metalloids, Group 1, Group 2, Group13, Group15, and Group 17.

Embodiment 33. A method of any of Embodiments 28 through 31 wherein the solid carbon precursor comprises a porous carbon, the method further comprising heating to melt the lithium precursor and intrude the lithium precursor within the pores of the porous carbon, and react the lithium precursor with the porous carbon to form the lithium carbide compound.

Embodiment 34. A method of Embodiment 28 wherein the heating of the precursors is achieved by spark plasma sintering.

Embodiment 35. A method of manufacturing a lithium carbide compound, the method comprising:
a. preparing an organolithium solution comprising an ethereal solvent, a lithium metal, and an arene;
b. introducing an alkyne-containing hydrocarbon to react to with the organolithium solution to form the lithium carbide compound;
wherein the reaction occurs under an inert environment.

Embodiment 36. The method of Embodiment 35, wherein the alkyne-containing hydrocarbon is a gas.

Embodiment 37. The method of Embodiment 35, wherein the alkyne-containing hydrocarbon is acetylene.

Embodiment 38. The method of Embodiment 35, wherein the alkyne-containing hydrocarbon is diethynylbenzene, triethynylbenzene, or combinations thereof.

Embodiment 39. A method of any of Embodiments 35 through 38 further comprising mixing a third precursor comprising an element selected from transition metals, metalloids, Group 1, Group 2, Group13, Group15, and Group 17.

Embodiment 40. A method of manufacturing a lithium carbide compound, the method comprising:
a. preparing a carbon precursor in an inert environment;
b. heating the carbon precursor to a temperature at or above the boiling point of the lithium precursor; and
c. introducing a vapor phase lithium precursor at or above the boiling point of the lithium precursor to react with the carbon precursor to form the lithium carbide compound.

Embodiment 41. A method of manufacturing a lithium carbide compound, the method comprising:
d. preparing a porous carbon precursor in an inert environment;
e. heating the carbon precursor to a temperature at or above the boiling point of the lithium precursor; and
f. introducing a vapor phase lithium precursor at or above the boiling point of the lithium precursor to infiltrate the porosity of the porous carbon and react with the porous carbon precursor to form the lithium carbide compound.

Embodiment 42. The method of Embodiment 40 and Embodiment 41, wherein the lithium precursor comprises an organolithium compound such as lithium bis(trimethylsilyl)amide.

Embodiment 43. The method of Embodiment 40 and Embodiment 41, wherein the lithium precursor comprises lithium metal and the inert environment is comprised of a high vacuum atmosphere.

Embodiment 44. A method of any of Embodiments 40 through 43 further comprising mixing a third precursor comprising an element selected from transition metals, metalloids, Group 1, Group 2, Group13, Group15, and Group 17.

Embodiment 45. The process of any of Embodiment 28 through Embodiment 44 wherein the carbon precursor comprises a plurality of particles comprising a Dv50 between 0.1 and 50 microns.

Embodiment 46. The process of Embodiment 33 or Embodiment 41 wherein the porous carbon precursor comprises a pore volume greater than 0.5 cm³/g.

Embodiment 47. The process of Embodiment 33 or Embodiment 41 wherein the porous carbon scaffold comprises micropores and mesopores.

Embodiment 48. A reducing agent comprising:
a solid lithium carbide compound comprising:
a plurality of lithium carbide particles comprising a Dv50 between 0.1 and 50 microns and a surface area less than 20 m2/g; and
wherein individual particles of the plurality of particles comprise a compound having a stoichiometry of LixCyMz, wherein x ranges from greater than 0 to 2, y is 2, and z ranges from 0 to 2; and wherein M is an element selected from one of a transition metal, metalloid, Group 1 element, Group 2 element, Group13 element, Group15 element, or Group 17 element.

Embodiment 49. A method for storing acetylene comprising:
hydrolyzing a lithium carbide material having a surface area and particle size to produce acetylene; and
wherein the surface area and particle size area are proportional to a production rate of acetylene produced from the hydrolyzing; and
wherein the lithium carbide material comprises a plurality of lithium carbide particles comprising a Dv50 between 0.1 and 50 microns and a surface area less than 20 m2/g; and wherein individual particles of the plurality of particles comprising a compound having a stoichiometry of LixCyMz, wherein x ranges from greater than 0 to 2, y is 2, and z ranges from 0 to 2; and wherein M is an element selected from one of a transition metal, metalloid, Group 1 element, Group 2 element, Group13 element, Group15 element, or Group 17 element.

Embodiment 50. A method for determining age of a carbon material comprising:
reacting the carbon material or a reaction byproduct of the carbon material with a lithium carbide material;
reacting the reaction byproduct with a second intermediate material to produce a detection species;
measuring concentration of the detection species; and
correlating the concentration of the detection species to the age of the carbon material.

Embodiment 51. The method of Embodiment 50, wherein the lithium carbide material comprises a plurality of lithium carbide particles comprising a Dv50 between 0.1 and 50 microns and a surface area less than 20 m2/g; and wherein individual particles of the plurality of particles comprising a compound having a stoichiometry of LixCyMz, wherein x ranges from greater than 0 to 2, y is 2, and z ranges from 0 to 2; and wherein M is an element selected from one of a transition metal, metalloid, Group 1 element, Group 2 element, Group13 element, Group15 element, or Group 17 element.

Embodiment 52. The method of the any of the previous Embodiments, wherein the byproduct is acetylene or benzene.

Embodiment 53. The method of the any of the previous Embodiments, wherein the second intermediate material is water and the reaction byproduct is acetylene.

Embodiment 54. The method of the any of the previous Embodiments, wherein the lithium compound is selected from lithium metal, lithium salts selected comprising lithium oxides, lithium nitrides, lithium halides, organolithium compounds, lithium alloys, and combinations thereof.

Embodiment 55. The method of the any of the previous Embodiments, further comprising synthesizing a lithium carbide compound using one of a melt infusion method, a vapor phase method, a reduction of lithium salts method, or an electrochemical method.

From the foregoing it will be appreciated that, although specific embodiments of the disclosure have been described herein for purposes of illustration, various modifications may be made without deviating from the spirit and scope of the disclosure.

1. A reducing agent comprising:
a solid lithium carbide compound comprising:
a plurality of lithium carbide particles comprising a Dv50 between 0.1 and 50 microns and a surface area less than 20 m²/g; and
wherein individual particles of the plurality of particles comprise a compound having a stoichiometry of LiₓC_{y}M_{z}, wherein x ranges from greater than 0 to 2, y is 2, and z ranges from 0 to 2; and wherein M is an element selected from one of a transition metal, metalloid, Group 1 element, Group 2 element, Group13 element, Group15 element, or Group 17 element.

2. A method for storing acetylene comprising:
hydrolyzing a lithium carbide material having a surface area and particle size to produce acetylene; and
wherein the surface area and particle size area are proportional to a production rate of acetylene produced from the hydrolyzing; and
wherein the lithium carbide material comprises a plurality of lithium carbide particles comprising a Dv50 between 0.1 and 50 microns and a surface area less than 20 m²/g; and wherein individual particles of the plurality of particles comprising a compound having a stoichiometry of LiₓC_{y}M_{z}, wherein x ranges from greater than 0 to 2, y is 2, and z ranges from 0 to 2; and wherein M is an element selected from one of a transition metal, metalloid, Group 1 element, Group 2 element, Group13 element, Group15 element, or Group 17 element.

3. A method for determining age of a carbon material comprising:
reacting the carbon material or a reaction byproduct of the carbon material with a lithium carbide material;
reacting the reaction byproduct with a second intermediate material to produce a detection species;
measuring concentration of the detection species; and
correlating the concentration of the detection species to the age of the carbon material.

4. The method of claim 3, wherein the lithium carbide material comprises a plurality of lithium carbide particles comprising a Dv50 between 0.1 and 50 microns and a surface area less than 20 m²/g; and wherein individual particles of the plurality of particles comprising a compound having a stoichiometry of LiₓC_{y}M_{z}, wherein x ranges from greater than 0 to 2, y is 2, and z ranges from 0 to 2; and wherein M is an element selected from one of a transition metal, metalloid, Group 1 element, Group 2 element, Group13 element, Group15 element, or Group 17 element.

5. The method of claim 3, wherein the byproduct is acetylene or benzene.

6. The method of claim 3, wherein the second intermediate material is water and the reaction byproduct is acetylene.

7. The method of claim 4, wherein the lithium compound is selected from lithium metal, lithium salts selected comprising lithium oxides, lithium nitrides, lithium halides, organolithium compounds, lithium alloys, and combinations thereof.

8. The method of claim 3, further comprising synthesizing a lithium carbide compound using one of a melt infusion method, a vapor phase method, a reduction of lithium salts method, or an electrochemical method.
